(19) United States
(12) Patent Application Publication
(10) Pub. No.: US 2025/0235153 A1
(43) Pub. Date: Jul. 24, 2025

(54) TECHNIQUES FOR STABLE PRESSURE DIFFERENCE FOR OPTICAL MEASUREMENTS USING A WEARABLE DEVICE

(71) Applicant: Oura Health Oy, Oulu (FI)

(72) Inventors: Jouni Juhani Huopana, Oulu (FI); Jukka Tapani Mäkinen, Oulu (FI)

(21) Appl. No.: 19/033,451

(22) Filed: Jan. 21, 2025

Related U.S. Application Data
(60) Provisional application No. 63/624,236, filed on Jan. 23, 2024.

(51) Int. Cl. A61B 5/00; A61B 5/024

(52) U.S. Cl. CPC A61B 5/4812; A61B 5/02427; A61B 5/02438; A61B 5/681; A61B 5/6843; A61B 5/7221

(57) ABSTRACT

Methods, systems, and devices for optical measurements using a wearable device are described. A wearable device may include multiple differently-sized protrusions to collect more accurate physiological data. The differently-sized protrusions may result in a constant pressure difference between contact pressures at the respective protrusions and the tissue of the user, while an overall magnitude of the pressures between each protrusion and the tissue may vary. Techniques described herein may enable the wearable device to determine whether changes in photoplethysmogram (PPG) signals are due to pressure effects or to biological changes using the constant pressure difference without the use of contact pressure sensors. The wearable device may determine which of the multiple protrusions may be associated with a highest quality of measurement data and may therefore select to use sensors (e.g., light-emitting diodes, photodetectors) within specific protrusions to perform the measurements.

### CROSS REFERENCE

The present Application for Patent claims the benefit of U.S. Provisional Patent Application No. 63/624,236 by HUOPANA et al., entitled “TECHNIQUES FOR STABLE PRESSURE DIFFERENCE FOR OPTICAL MEASUREMENTS USING A WEARABLE DEVICE,” filed Jan. 23, 2024, assigned to the assignee hereof, and expressly incorporated by reference herein.

### FIELD OF TECHNOLOGY

The following relates to wearable devices and data processing, including techniques for leveraging stable pressure differences for optical measurements using a wearable device.

### BACKGROUND

Some wearable devices may be configured to collect data from users, including heart rate data, temperature data, blood oxygen saturation data, and the like. The wearable devices may use light-transmitting and light-receiving components to collect the data. However, a quality of data collected by the light-transmitting and light-receiving components may vary depending on a contact pressure between a wearable device and the tissue of a user.

### BRIEF DESCRIPTION OF THE DRAWINGS

FIG. 1 illustrates an example of a system that supports techniques for leveraging stable pressure differences for optical measurements using a wearable device in accordance with aspects of the present disclosure.

FIG. 2 illustrates an example of a system that supports techniques for leveraging stable pressure differences for optical measurements using a wearable device in accordance with aspects of the present disclosure.

FIG. 3 shows an example of a process flow that supports techniques for leveraging stable pressure differences for optical measurements using a wearable device in accordance with aspects of the present disclosure.

FIG. 4 shows an example of a sensor diagram that supports techniques for leveraging stable pressure differences for optical measurements using a wearable device in accordance with aspects of the present disclosure.

FIG. 5 shows an example of a flowchart that supports techniques for leveraging stable pressure differences for optical measurements using a wearable device in accordance with aspects of the present disclosure.

### DETAILED DESCRIPTION

Wearable devices can be configured to collect physiological data from users to provide users with more information regarding their sleep patterns and overall health. Physiological data collected from wearable devices may include heart rate data, temperature data, respiration rate data, blood oxygen saturation data, and the like. In some cases, the wearable devices may include one or more protrusions (e.g., domes) that house one or more light emitting diodes (LEDs) or light sensors such as photodetectors (PDs) that may transmit and measure light pulses to collect the physiological data. However, in some cases, the light pulses may penetrate tissue of the user differently depending on a pressure applied to the measurement area of the tissue (e.g., from the dome-shaped protrusions). Thus, variations in pressure between the wearable device and the tissue (due to tightness of the wearable device, swelling, aging, etc.) may cause variation in the collected physiological data, which may result in less accurate measurements and may reduce user satisfaction.

Accordingly, as described herein, a wearable device may include multiple differently-sized protrusions (e.g., dome-shaped protrusions) to collect more accurate physiological data when such pressure differences are present. In particular, light associated with the same wavelength may penetrate the tissue of the user to different penetration depths when emitted from protrusions of varying height (due to differences in contact pressure with the user's tissue at the differently-sized protrusions). For example, the wearable device may include a first dome-shaped protrusion with a first height/size and a second dome-shaped protrusion with a second height/size (e.g., larger than the first size). The differently sized protrusions may result in a constant pressure difference between contact pressures at the respective domes and the tissue of the user, while an overall magnitude of the pressures between each protrusion and the tissue may vary. For example, if a finger of the user experiences swelling, the overall contact pressures at the respective domes may increase, while the difference in pressure across each of the differently sized domes may remain constant (or relatively/substantially constant).

Using the constant pressure difference, techniques described herein may enable the wearable device to determine whether changes in photoplethysmogram (PPG) signals are due to pressure effects (e.g., due to the finger swelling or some other pressure effect), or due to actual biological changes, such as changes in the user's blood pressure or blood oxygen saturation. Moreover, techniques described herein may enable wearable devices to analyze such pressure effects without the use of contact pressure sensors (such as piezoelectric sensors). For example, a PPG signal strength/quality may change from one day to the next. A conventional wearable device may be unable to determine whether the change in PPG signal strength/quality is due to an actual physiological change in the user (e.g., change in blood oxygen saturation), or due to the user's finger's swelling, resulting in increased contact pressure between the wearable device and the tissue of the user. Comparatively, by using PPG data collected using differently-sized protrusions with a constant pressure difference across the protrusions, a wearable device described herein may be able to determine whether or not a contact pressure between the wearable device and the tissue of the user has changed. As such, techniques described herein may enable the wearable device to determine whether or not changes in PPG signal strength/quality are due to changes in contact pressure, or due to actual physiological changes in the user.

Further, the wearable device may determine which of the multiple dome-shaped protrusions (e.g., what level of contact pressure) may be associated with the highest quality of measurement data. The wearable device may therefore select to use LEDs or PDs within specific dome-shaped protrusions (e.g., at the determined contact pressure) to perform the measurements, thereby resulting in collected physiological data that exhibits a higher quality, accuracy, and/or reliability as compared with some other conventional wearable devices.

Aspects of the disclosure are initially described in the context of systems supporting physiological data collection from users via wearable devices. Aspects of the disclosure are further illustrated by and described with reference to process flow diagrams that relate to techniques for leveraging stable pressure differences for optical measurements using a wearable device.

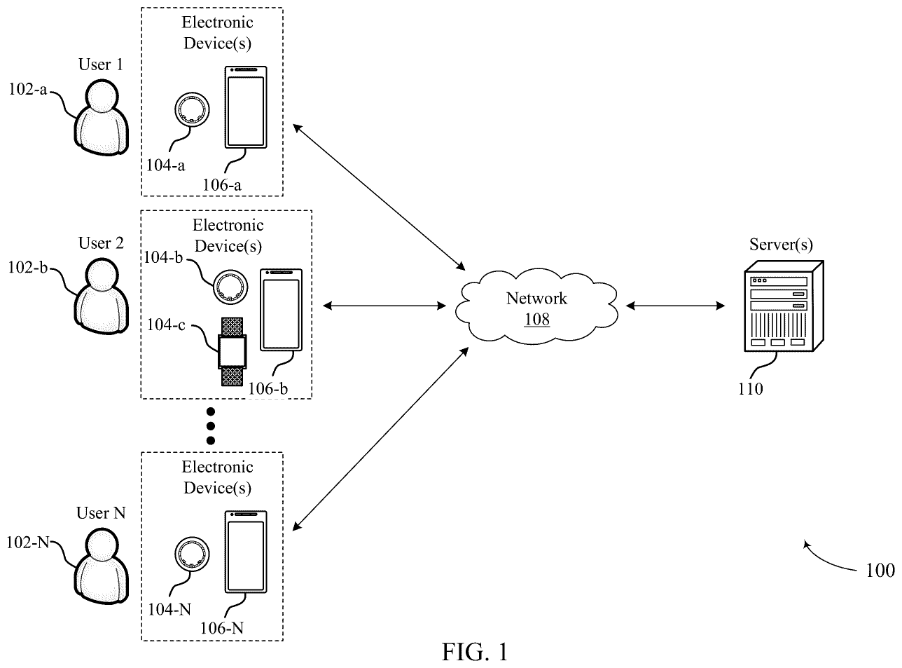

FIG. 1 illustrates an example of a system 100 that supports techniques for leveraging stable pressure differences for optical measurements using a wearable device in accordance with aspects of the present disclosure. The system 100 includes a plurality of electronic devices (e.g., wearable devices 104, user devices 106) that may be worn and/or operated by one or more users 102. The system 100 further includes a network 108 and one or more servers 110.

The electronic devices may include any electronic devices known in the art, including wearable devices 104 (e.g., ring wearable devices, watch wearable devices, etc.), user devices 106 (e.g., smartphones, laptops, tablets). The electronic devices associated with the respective users 102 may include one or more of the following functionalities: 1) measuring physiological data, 2) storing the measured data, 3) processing the data, 4) providing outputs (e.g., via GUIs) to a user 102 based on the processed data, and 5) communicating data with one another and/or other computing devices. Different electronic devices may perform one or more of the functionalities.

Example wearable devices 104 may include wearable computing devices, such as a ring computing device (hereinafter “ring”) configured to be worn on a user's 102 finger, a wrist computing device (e.g., a smart watch, fitness band, or bracelet) configured to be worn on a user's 102 wrist, and/or a head mounted computing device (e.g., glasses/goggles). Wearable devices 104 may also include bands, straps (e.g., flexible or inflexible bands or straps), stick-on sensors, and the like, that may be positioned in other locations, such as bands around the head (e.g., a forehead headband), arm (e.g., a forearm band and/or bicep band), and/or leg (e.g., a thigh or calf band), behind the car, under the armpit, and the like. Wearable devices 104 may also be attached to, or included in, articles of clothing. For example, wearable devices 104 may be included in pockets and/or pouches on clothing. As another example, wearable device 104 may be clipped and/or pinned to clothing, or may otherwise be maintained within the vicinity of the user 102. Example articles of clothing may include, but are not limited to, hats, shirts, gloves, pants, socks, outerwear (e.g., jackets), and undergarments. In some implementations, wearable devices 104 may be included with other types of devices such as training/sporting devices that are used during physical activity. For example, wearable devices 104 may be attached to, or included in, a bicycle, skis, a tennis racket, a golf club, and/or training weights.

Much of the present disclosure may be described in the context of a ring wearable device 104. Accordingly, the terms “ring 104,” “wearable device 104,” and like terms, may be used interchangeably, unless noted otherwise herein. However, the use of the term “ring 104” is not to be regarded as limiting, as it is contemplated herein that aspects of the present disclosure may be performed using other wearable devices (e.g., watch wearable devices, necklace wearable device, bracelet wearable devices, earring wearable devices, anklet wearable devices, and the like).

In some aspects, user devices 106 may include handheld mobile computing devices, such as smartphones and tablet computing devices. User devices 106 may also include personal computers, such as laptop and desktop computing devices. Other example user devices 106 may include server computing devices that may communicate with other electronic devices (e.g., via the Internet). In some implementations, computing devices may include medical devices, such as external wearable computing devices (e.g., Holter monitors). Medical devices may also include implantable medical devices, such as pacemakers and cardioverter defibrillators. Other example user devices 106 may include home computing devices, such as internet of things (IoT) devices (e.g., IoT devices), smart televisions, smart speakers, smart displays (e.g., video call displays), hubs (e.g., wireless communication hubs), security systems, smart appliances (e.g., thermostats and refrigerators), and fitness equipment.

Some electronic devices (e.g., wearable devices 104, user devices 106) may measure physiological parameters of respective users 102, such as photoplethysmography waveforms, continuous skin temperature, a pulse waveform, respiration rate, heart rate, heart rate variability (HRV), actigraphy, galvanic skin response, pulse oximetry, blood oxygen saturation (SpO2), blood sugar levels (e.g., glucose metrics), and/or other physiological parameters. Some electronic devices that measure physiological parameters may also perform some/all of the calculations described herein. Some electronic devices may not measure physiological parameters, but may perform some/all of the calculations described herein. For example, a ring (e.g., wearable device 104), mobile device application, or a server computing device may process received physiological data that was measured by other devices.

In some implementations, a user 102 may operate, or may be associated with, multiple electronic devices, some of which may measure physiological parameters and some of which may process the measured physiological parameters. In some implementations, a user 102 may have a ring (e.g., wearable device 104) that measures physiological parameters. The user 102 may also have, or be associated with, a user device 106 (e.g., mobile device, smartphone), where the wearable device 104 and the user device 106 are communicatively coupled to one another. In some cases, the user device 106 may receive data from the wearable device 104 and perform some/all of the calculations described herein. In some implementations, the user device 106 may also measure physiological parameters described herein, such as motion/activity parameters.

For example, as illustrated in FIG. 1, a first user 102-a (User 1) may operate, or may be associated with, a wearable device 104-a (e.g., ring 104-a) and a user device 106-a that may operate as described herein. In this example, the user device 106-a associated with user 102-a may process/store physiological parameters measured by the ring 104-a. Comparatively, a second user 102-b (User 2) may be associated with a ring 104-b, a watch wearable device 104-c (e.g., watch 104-c), and a user device 106-b, where the user device 106-b associated with user 102-b may process/store physiological parameters measured by the ring 104-b and/or the watch 104-c. Moreover, an nth user 102-n (User N) may be associated with an arrangement of electronic devices described herein (e.g., ring 104-n, user device 106-n). In some aspects, wearable devices 104 (e.g., rings 104, watches 104) and other electronic devices may be communicatively coupled to the user devices 106 of the respective users 102 via Bluetooth, Wi-Fi, and other wireless protocols. Moreover, in some cases, the wearable device 104 and the user device 106 may be included within (or make up) the same device. For example, in some cases, the wearable device 104 may be configured to execute an application associated with the wearable device 104, and may be configured to display data via a GUI.

In some implementations, the rings 104 (e.g., wearable devices 104) of the system 100 may be configured to collect physiological data from the respective users 102 based on arterial blood flow within the user's finger. In particular, a ring 104 may utilize one or more light-emitting components, such as LEDs (e.g., red LEDs, green LEDs) that emit light on the palm-side of a user's finger to collect physiological data based on arterial blood flow within the user's finger. In general, the terms light-emitting components, light-emitting elements, and like terms, may include, but are not limited to, LEDs, micro LEDs, mini LEDs, laser diodes (LDs) (e.g., vertical cavity surface-emitting lasers (VCSELs), and the like.

In some cases, the system 100 may be configured to collect physiological data from the respective users 102 based on blood flow diffused into a microvascular bed of skin with capillaries and arterioles. For example, the system 100 may collect PPG data based on a measured amount of blood diffused into the microvascular system of capillaries and arterioles. In some implementations, the ring 104 may acquire the physiological data using a combination of both green and red LEDs. The physiological data may include any physiological data known in the art including, but not limited to, temperature data, accelerometer data (e.g., movement/motion data), heart rate data, HRV data, blood oxygen level data, or any combination thereof.

The use of both green and red LEDs may provide several advantages over other solutions, as red and green LEDs have been found to have their own distinct advantages when acquiring physiological data under different conditions (e.g., light/dark, active/inactive) and via different parts of the body, and the like. For example, green LEDs have been found to exhibit better performance during exercise. Moreover, using multiple LEDs (e.g., green and red LEDs) distributed around the ring 104 has been found to exhibit superior performance as compared to wearable devices that utilize LEDs that are positioned close to one another, such as within a watch wearable device. Furthermore, the blood vessels in the finger (e.g., arteries, capillaries) are more accessible via LEDs as compared to blood vessels in the wrist. In particular, arteries in the wrist are positioned on the bottom of the wrist (e.g., palm-side of the wrist), meaning only capillaries are accessible on the top of the wrist (e.g., back of hand side of the wrist), where wearable watch devices and similar devices are typically worn. As such, utilizing LEDs and other sensors within a ring 104 has been found to exhibit superior performance as compared to wearable devices worn on the wrist, as the ring 104 may have greater access to arteries (as compared to capillaries), thereby resulting in stronger signals and more valuable physiological data.

The electronic devices of the system 100 (e.g., user devices 106, wearable devices 104) may be communicatively coupled to one or more servers 110 via wired or wireless communication protocols. For example, as shown in FIG. 1, the electronic devices (e.g., user devices 106) may be communicatively coupled to one or more servers 110 via a network 108. The network 108 may implement transfer control protocol and internet protocol (TCP/IP), such as the Internet, or may implement other network 108 protocols. Network connections between the network 108 and the respective electronic devices may facilitate transport of data via email, web, text messages, mail, or any other appropriate form of interaction within a computer network 108. For example, in some implementations, the ring 104-a associated with the first user 102-a may be communicatively coupled to the user device 106-a, where the user device 106-a is communicatively coupled to the servers 110 via the network 108. In additional or alternative cases, wearable devices 104 (e.g., rings 104, watches 104) may be directly communicatively coupled to the network 108.

The system 100 may offer an on-demand database service between the user devices 106 and the one or more servers 110. In some cases, the servers 110 may receive data from the user devices 106 via the network 108, and may store and analyze the data. Similarly, the servers 110 may provide data to the user devices 106 via the network 108. In some cases, the servers 110 may be located at one or more data centers. The servers 110 may be used for data storage, management, and processing. In some implementations, the servers 110 may provide a web-based interface to the user device 106 via web browsers.

In some aspects, the system 100 may detect periods of time that a user 102 is asleep, and classify periods of time that the user 102 is asleep into one or more sleep stages (e.g., sleep stage classification). For example, as shown in FIG. 1, User 102-a may be associated with a wearable device 104-a (e.g., ring 104-a) and a user device 106-a. In this example, the ring 104-a may collect physiological data associated with the user 102-a, including temperature, heart rate, HRV, respiratory rate, and the like. In some aspects, data collected by the ring 104-a may be input to a machine learning classifier, where the machine learning classifier is configured to determine periods of time that the user 102-a is (or was) asleep. Moreover, the machine learning classifier may be configured to classify periods of time into different sleep stages, including an awake sleep stage, a rapid eye movement (REM) sleep stage, a light sleep stage (non-REM (NREM)), and a deep sleep stage (NREM). In some aspects, the classified sleep stages may be displayed to the user 102-a via a GUI of the user device 106-a. Sleep stage classification may be used to provide feedback to a user 102-a regarding the user's sleeping patterns, such as recommended bedtimes, recommended wake-up times, and the like. Moreover, in some implementations, sleep stage classification techniques described herein may be used to calculate scores for the respective user, such as Sleep Scores, Readiness Scores, and the like.

In some aspects, the system 100 may utilize circadian rhythm-derived features to further improve physiological data collection, data processing procedures, and other techniques described herein. The term circadian rhythm may refer to a natural, internal process that regulates an individual's sleep-wake cycle, that repeats approximately every 24 hours. In this regard, techniques described herein may utilize circadian rhythm adjustment models to improve physiological data collection, analysis, and data processing. For example, a circadian rhythm adjustment model may be input into a machine learning classifier along with physiological data collected from the user 102-a via the wearable device 104-a. In this example, the circadian rhythm adjustment model may be configured to “weight,” or adjust, physiological data collected throughout a user's natural, approximately 24-hour circadian rhythm. In some implementations, the system may initially start with a “baseline” circadian rhythm adjustment model, and may modify the baseline model using physiological data collected from each user 102 to generate tailored, individualized circadian rhythm adjustment models that are specific to each respective user 102.

In some aspects, the system 100 may utilize other biological rhythms to further improve physiological data collection, analysis, and processing by phase of these other rhythms. For example, if a weekly rhythm is detected within an individual's baseline data, then the model may be configured to adjust “weights” of data by day of the week. Biological rhythms that may require adjustment to the model by this method include: 1) ultradian (faster than a day rhythms, including sleep cycles in a sleep state, and oscillations from less than an hour to several hours periodicity in the measured physiological variables during wake state; 2) circadian rhythms; 3) non-endogenous daily rhythms shown to be imposed on top of circadian rhythms, as in work schedules; 4) weekly rhythms, or other artificial time periodicities exogenously imposed (e.g., in a hypothetical culture with 12 day “weeks,” 12 day rhythms could be used); 5) multi-day ovarian rhythms in women and spermatogenesis rhythms in men; 6) lunar rhythms (relevant for individuals living with low or no artificial lights); and 7) seasonal rhythms.

The biological rhythms are not always stationary rhythms. For example, many women experience variability in ovarian cycle length across cycles, and ultradian rhythms are not expected to occur at exactly the same time or periodicity across days even within a user. As such, signal processing techniques sufficient to quantify the frequency composition while preserving temporal resolution of these rhythms in physiological data may be used to improve detection of these rhythms, to assign phase of each rhythm to each moment in time measured, and to thereby modify adjustment models and comparisons of time intervals. The biological rhythm-adjustment models and parameters can be added in linear or non-linear combinations as appropriate to more accurately capture the dynamic physiological baselines of an individual or group of individuals.

In some aspects, the respective devices of the system 100 may support techniques for a wearable device 104 to use multiple protrusions of different heights/sizes to collect more accurate physical data. For example, the wearable device 104 (e.g., a wearable ring device, a wrist-worn wearable device) may use the multiple differently-sized dome-shaped protrusions to collect more accurate physiological data when pressure differences are present due to swelling, aging, tightness of the wearable device 104, and so on. In particular, light associated with the same wavelength may penetrate tissue of a user 102 to different penetration depths when emitted from protrusions of varying height. For example, the wearable device 104 may include a first dome-shaped protrusion with a first height and a second dome-shaped protrusion with a second height (e.g., larger than the first height). The differently-sized protrusions may result in a constant pressure difference between contact pressures at the respective domes and the tissue of the user while an overall magnitude of the pressures between each protrusion and the tissue may vary.

Using the constant pressure difference, techniques described herein may enable the wearable device 104 to determine whether changes in PPG signals are due to pressure effects (e.g., due to the finger swelling or some other pressure effect), or due to actual biological changes. Additionally, or alternatively, the wearable device 104 may determine which of the multiple dome-shaped protrusions (e.g., what level of contact pressure) may be associated with a highest quality of measurement data. The wearable device 104 may therefore select to use LEDs and/or PDs within specific dome-shaped protrusions (e.g., at the determined contact pressure) to perform the measurements.

It should be appreciated by a person skilled in the art that one or more aspects of the disclosure may be implemented in a system 100 to additionally, or alternatively, solve other problems than those described above. Furthermore, aspects of the disclosure may provide technical improvements to “conventional” systems or processes as described herein. However, the description and appended drawings only include example technical improvements resulting from implementing aspects of the disclosure, and accordingly do not represent all of the technical improvements provided within the scope of the claims.

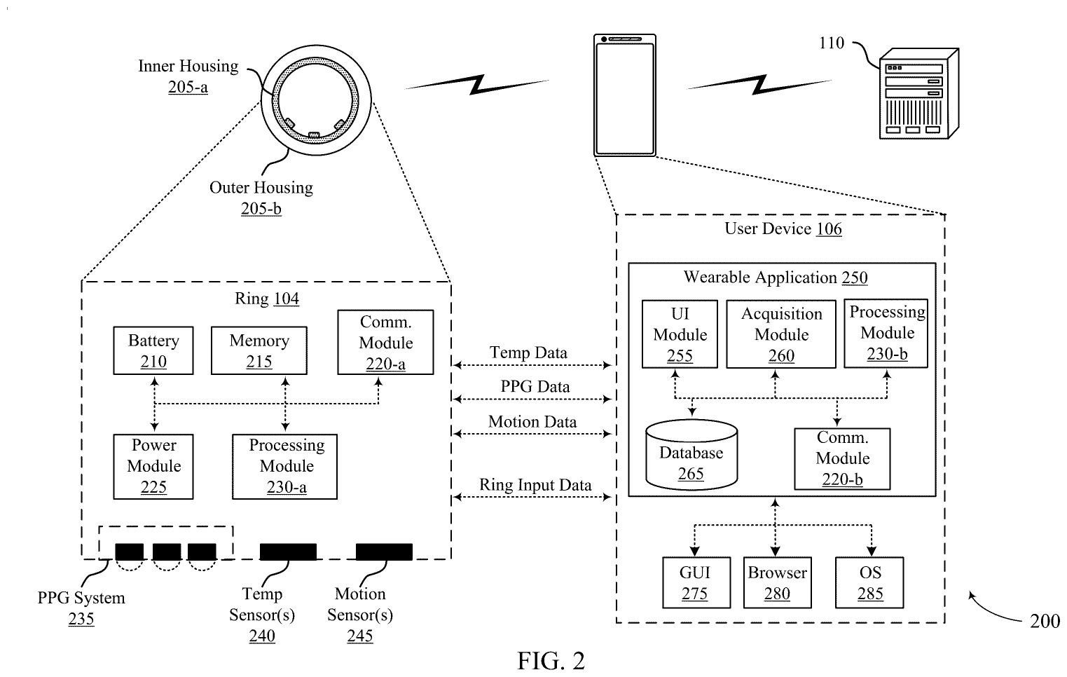

FIG. 2 illustrates an example of a system 200 that supports techniques for leveraging stable pressure differences for optical measurements using a wearable device in accordance with aspects of the present disclosure. The system 200 may implement, or be implemented by, system 100. In particular, system 200 illustrates an example of a ring 104 (e.g., wearable device 104), a user device 106, and a server 110, as described with reference to FIG. 1.

In some aspects, the ring 104 may be configured to be worn around a user's finger, and may determine one or more user physiological parameters when worn around the user's finger. Example measurements and determinations may include, but are not limited to, user skin temperature, pulse waveforms, respiratory rate, heart rate, HRV, blood oxygen levels (SpO2), blood sugar levels (e.g., glucose metrics), and the like.

The system 200 further includes a user device 106 (e.g., a smartphone) in communication with the ring 104. For example, the ring 104 may be in wireless and/or wired communication with the user device 106. In some implementations, the ring 104 may send measured and processed data (e.g., temperature data, PPG data, motion/accelerometer data, ring input data, and the like) to the user device 106. The user device 106 may also send data to the ring 104, such as ring 104 firmware/configuration updates. The user device 106 may process data. In some implementations, the user device 106 may transmit data to the server 110 for processing and/or storage.

The ring 104 may include a housing 205 that may include an inner housing 205-a and an outer housing 205-b. In some aspects, the housing 205 of the ring 104 may store or otherwise include various components of the ring including, but not limited to, device electronics, a power source (e.g., battery 210, and/or capacitor), one or more substrates (e.g., printable circuit boards) that interconnect the device electronics and/or power source, and the like. The device electronics may include device modules (e.g., hardware/software), such as: a processing module 230-a, a memory 215, a communication module 220-a, a power module 225, and the like. The device electronics may also include one or more sensors. Example sensors may include one or more temperature sensors 240, a PPG sensor assembly (e.g., PPG system 235), and one or more motion sensors 245.

The sensors may include associated modules (not illustrated) configured to communicate with the respective components/modules of the ring 104, and generate signals associated with the respective sensors. In some aspects, each of the components/modules of the ring 104 may be communicatively coupled to one another via wired or wireless connections. Moreover, the ring 104 may include additional and/or alternative sensors or other components that are configured to collect physiological data from the user, including light sensors (e.g., LEDs), oximeters, and the like.

The ring 104 shown and described with reference to FIG. 2 is provided solely for illustrative purposes. As such, the ring 104 may include additional or alternative components as those illustrated in FIG. 2. Other rings 104 that provide functionality described herein may be fabricated. For example, rings 104 with fewer components (e.g., sensors) may be fabricated. In a specific example, a ring 104 with a single temperature sensor 240 (or other sensor), a power source, and device electronics configured to read the single temperature sensor 240 (or other sensor) may be fabricated. In another specific example, a temperature sensor 240 (or other sensor) may be attached to a user's finger (e.g., using adhesives, wraps, clamps, spring loaded clamps, etc.). In this case, the sensor may be wired to another computing device, such as a wrist worn computing device that reads the temperature sensor 240 (or other sensor). In other examples, a ring 104 that includes additional sensors and processing functionality may be fabricated.

The housing 205 may include one or more housing 205 components. The housing 205 may include an outer housing 205-b component (e.g., a shell) and an inner housing 205-a component (e.g., a molding). The housing 205 may include additional components (e.g., additional layers) not explicitly illustrated in FIG. 2. For example, in some implementations, the ring 104 may include one or more insulating layers that electrically insulate the device electronics and other conductive materials (e.g., electrical traces) from the outer housing 205-b (e.g., a metal outer housing 205-b). The housing 205 may provide structural support for the device electronics, battery 210, substrate(s), and other components. For example, the housing 205 may protect the device electronics, battery 210, and substrate(s) from mechanical forces, such as pressure and impacts. The housing 205 may also protect the device electronics, battery 210, and substrate(s) from water and/or other chemicals.

The outer housing 205-b may be fabricated from one or more materials. In some implementations, the outer housing 205-b may include a metal, such as titanium, that may provide strength and abrasion resistance at a relatively light weight. The outer housing 205-b may also be fabricated from other materials, such polymers. In some implementations, the outer housing 205-b may be protective as well as decorative.

The inner housing 205-a may be configured to interface with the user's finger. The inner housing 205-a may be formed from a polymer (e.g., a medical grade polymer) or other material. In some implementations, the inner housing 205-a may be transparent. For example, the inner housing 205-a may be transparent to light emitted by the PPG LEDs. In some implementations, the inner housing 205-a component may be molded onto the outer housing 205-b. For example, the inner housing 205-a may include a polymer that is molded (e.g., injection molded) to fit into an outer housing 205-b metallic shell.

The ring 104 may include one or more substrates (not illustrated). The device electronics and battery 210 may be included on the one or more substrates. For example, the device electronics and battery 210 may be mounted on one or more substrates. Example substrates may include one or more printed circuit boards (PCBs), such as flexible PCB (e.g., polyimide). In some implementations, the electronics/battery 210 may include surface mounted devices (e.g., surface-mount technology (SMT) devices) on a flexible PCB. In some implementations, the one or more substrates (e.g., one or more flexible PCBs) may include electrical traces that provide electrical communication between device electronics. The electrical traces may also connect the battery 210 to the device electronics.

The device electronics, battery 210, and substrates may be arranged in the ring 104 in a variety of ways. In some implementations, one substrate that includes device electronics may be mounted along the bottom of the ring 104 (e.g., the bottom half), such that the sensors (e.g., PPG system 235, temperature sensors 240, motion sensors 245, and other sensors) interface with the underside of the user's finger. In these implementations, the battery 210 may be included along the top portion of the ring 104 (e.g., on another substrate).

The various components/modules of the ring 104 represent functionality (e.g., circuits and other components) that may be included in the ring 104. Modules may include any discrete and/or integrated electronic circuit components that implement analog and/or digital circuits capable of producing the functions attributed to the modules herein. For example, the modules may include analog circuits (e.g., amplification circuits, filtering circuits, analog/digital conversion circuits, and/or other signal conditioning circuits). The modules may also include digital circuits (e.g., combinational or sequential logic circuits, memory circuits etc.).

The memory 215 (memory module) of the ring 104 may include any volatile, non-volatile, magnetic, or electrical media, such as a random access memory (RAM), read-only memory (ROM), non-volatile RAM (NVRAM), electrically-erasable programmable ROM (EEPROM), flash memory, or any other memory device. The memory 215 may store any of the data described herein. For example, the memory 215 may be configured to store data (e.g., motion data, temperature data, PPG data) collected by the respective sensors and PPG system 235. Furthermore, memory 215 may include instructions that, when executed by one or more processing circuits, cause the modules to perform various functions attributed to the modules herein. The device electronics of the ring 104 described herein are only example device electronics. As such, the types of electronic components used to implement the device electronics may vary based on design considerations.

The functions attributed to the modules of the ring 104 described herein may be embodied as one or more processors, hardware, firmware, software, or any combination thereof. Depiction of different features as modules is intended to highlight different functional aspects and does not necessarily imply that such modules must be realized by separate hardware/software components. Rather, functionality associated with one or more modules may be performed by separate hardware/software components or integrated within common hardware/software components.

The processing module 230-a of the ring 104 may include one or more processors (e.g., processing units), microcontrollers, digital signal processors, systems on a chip (SOCs), and/or other processing devices. The processing module 230-a communicates with the modules included in the ring 104. For example, the processing module 230-a may transmit/receive data to/from the modules and other components of the ring 104, such as the sensors. As described herein, the modules may be implemented by various circuit components. Accordingly, the modules may also be referred to as circuits (e.g., a communication circuit and power circuit).

The processing module 230-a may communicate with the memory 215. The memory 215 may include computer-readable instructions that, when executed by the processing module 230-a, cause the processing module 230-a to perform the various functions attributed to the processing module 230-a herein. In some implementations, the processing module 230-a (e.g., a microcontroller) may include additional features associated with other modules, such as communication functionality provided by the communication module 220-a (e.g., an integrated Bluetooth Low Energy transceiver) and/or additional onboard memory 215.

The communication module 220-a may include circuits that provide wireless and/or wired communication with the user device 106 (e.g., communication module 220-b of the user device 106). In some implementations, the communication modules 220-a, 220-b may include wireless communication circuits, such as Bluetooth circuits and/or Wi-Fi circuits. In some implementations, the communication modules 220-a, 220-b can include wired communication circuits, such as Universal Serial Bus (USB) communication circuits. Using the communication module 220-a, the ring 104 and the user device 106 may be configured to communicate with each other. The processing module 230-a of the ring may be configured to transmit/receive data to/from the user device 106 via the communication module 220-a. Example data may include, but is not limited to, motion data, temperature data, pulse waveforms, heart rate data, HRV data, PPG data, and status updates (e.g., charging status, battery charge level, and/or ring 104 configuration settings). The processing module 230-a of the ring may also be configured to receive updates (e.g., software/firmware updates) and data from the user device 106.

The ring 104 may include a battery 210 (e.g., a rechargeable battery 210). An example battery 210 may include a Lithium-Ion or Lithium-Polymer type battery 210, although a variety of battery 210 options are possible. The battery 210 may be wirelessly charged. In some implementations, the ring 104 may include a power source other than the battery 210, such as a capacitor. The power source (e.g., battery 210 or capacitor) may have a curved geometry that matches the curve of the ring 104. In some aspects, a charger or other power source may include additional sensors that may be used to collect data in addition to, or that supplements, data collected by the ring 104 itself. Moreover, a charger or other power source for the ring 104 may function as a user device 106, in which case the charger or other power source for the ring 104 may be configured to receive data from the ring 104, store and/or process data received from the ring 104, and communicate data between the ring 104 and the servers 110.

In some aspects, the ring 104 includes a power module 225 that may control charging of the battery 210. For example, the power module 225 may interface with an external wireless charger that charges the battery 210 when interfaced with the ring 104. The charger may include a datum structure that mates with a ring 104 datum structure to create a specified orientation with the ring 104 during charging. The power module 225 may also regulate voltage(s) of the device electronics, regulate power output to the device electronics, and monitor the state of charge of the battery 210. In some implementations, the battery 210 may include a protection circuit module (PCM) that protects the battery 210 from high current discharge, over voltage during charging, and under voltage during discharge. The power module 225 may also include electro-static discharge (ESD) protection.

The one or more temperature sensors 240 may be electrically coupled to the processing module 230-a. The temperature sensor 240 may be configured to generate a temperature signal (e.g., temperature data) that indicates a temperature read or sensed by the temperature sensor 240. The processing module 230-a may determine a temperature of the user in the location of the temperature sensor 240. For example, in the ring 104, temperature data generated by the temperature sensor 240 may indicate a temperature of a user at the user's finger (e.g., skin temperature). In some implementations, the temperature sensor 240 may contact the user's skin. In other implementations, a portion of the housing 205 (e.g., the inner housing 205-a) may form a barrier (e.g., a thin, thermally conductive barrier) between the temperature sensor 240 and the user's skin. In some implementations, portions of the ring 104 configured to contact the user's finger may have thermally conductive portions and thermally insulative portions. The thermally conductive portions may conduct heat from the user's finger to the temperature sensors 240. The thermally insulative portions may insulate portions of the ring 104 (e.g., the temperature sensor 240) from ambient temperature.

In some implementations, the temperature sensor 240 may generate a digital signal (e.g., temperature data) that the processing module 230-a may use to determine the temperature. As another example, in cases where the temperature sensor 240 includes a passive sensor, the processing module 230-a (or a temperature sensor 240 module) may measure a current/voltage generated by the temperature sensor 240 and determine the temperature based on the measured current/voltage. Example temperature sensors 240 may include a thermistor, such as a negative temperature coefficient (NTC) thermistor, or other types of sensors including resistors, transistors, diodes, and/or other electrical/electronic components.

The processing module 230-a may sample the user's temperature over time. For example, the processing module 230-a may sample the user's temperature according to a sampling rate. An example sampling rate may include one sample per second, although the processing module 230-a may be configured to sample the temperature signal at other sampling rates that are higher or lower than one sample per second. In some implementations, the processing module 230-a may sample the user's temperature continuously throughout the day and night. Sampling at a sufficient rate (e.g., one sample per second) throughout the day may provide sufficient temperature data for analysis described herein.

The processing module 230-a may store the sampled temperature data in memory 215. In some implementations, the processing module 230-a may process the sampled temperature data. For example, the processing module 230-a may determine average temperature values over a period of time. In one example, the processing module 230-a may determine an average temperature value each minute by summing all temperature values collected over the minute and dividing by the number of samples over the minute. In a specific example where the temperature is sampled at one sample per second, the average temperature may be a sum of all sampled temperatures for one minute divided by sixty seconds. The memory 215 may store the average temperature values over time. In some implementations, the memory 215 may store average temperatures (e.g., one per minute) instead of sampled temperatures in order to conserve memory 215.

The sampling rate, which may be stored in memory 215, may be configurable. In some implementations, the sampling rate may be the same throughout the day and night. In other implementations, the sampling rate may be changed throughout the day/night. In some implementations, the ring 104 may filter/reject temperature readings, such as large spikes in temperature that are not indicative of physiological changes (e.g., a temperature spike from a hot shower). In some implementations, the ring 104 may filter/reject temperature readings that may not be reliable due to other factors, such as excessive motion during exercise (e.g., as indicated by a motion sensor 245).

The ring 104 (e.g., communication module) may transmit the sampled and/or average temperature data to the user device 106 for storage and/or further processing. The user device 106 may transfer the sampled and/or average temperature data to the server 110 for storage and/or further processing.

Although the ring 104 is illustrated as including a single temperature sensor 240, the ring 104 may include multiple temperature sensors 240 in one or more locations, such as arranged along the inner housing 205-a near the user's finger. In some implementations, the temperature sensors 240 may be stand-alone temperature sensors 240. Additionally, or alternatively, one or more temperature sensors 240 may be included with other components (e.g., packaged with other components), such as with the accelerometer and/or processor.

The processing module 230-a may acquire and process data from multiple temperature sensors 240 in a similar manner described with respect to a single temperature sensor 240. For example, the processing module 230 may individually sample, average, and store temperature data from each of the multiple temperature sensors 240. In other examples, the processing module 230-a may sample the sensors at different rates and average/store different values for the different sensors. In some implementations, the processing module 230-a may be configured to determine a single temperature based on the average of two or more temperatures determined by two or more temperature sensors 240 in different locations on the finger.

The temperature sensors 240 on the ring 104 may acquire distal temperatures at the user's finger (e.g., any finger). For example, one or more temperature sensors 240 on the ring 104 may acquire a user's temperature from the underside of a finger or at a different location on the finger. In some implementations, the ring 104 may continuously acquire distal temperature (e.g., at a sampling rate). Although distal temperature measured by a ring 104 at the finger is described herein, other devices may measure temperature at the same/different locations. In some cases, the distal temperature measured at a user's finger may differ from the temperature measured at a user's wrist or other external body location. Additionally, the distal temperature measured at a user's finger (e.g., a “shell” temperature) may differ from the user's core temperature. As such, the ring 104 may provide a useful temperature signal that may not be acquired at other internal/external locations of the body. In some cases, continuous temperature measurement at the finger may capture temperature fluctuations (e.g., small or large fluctuations) that may not be evident in core temperature. For example, continuous temperature measurement at the finger may capture minute-to-minute or hour-to-hour temperature fluctuations that provide additional insight that may not be provided by other temperature measurements elsewhere in the body.

The ring 104 may include a PPG system 235. The PPG system 235 may include one or more optical transmitters that transmit light. The PPG system 235 may also include one or more optical receivers that receive light transmitted by the one or more optical transmitters. An optical receiver may generate a signal (hereinafter “PPG” signal) that indicates an amount of light received by the optical receiver. The optical transmitters may illuminate a region of the user's finger. The PPG signal generated by the PPG system 235 may indicate the perfusion of blood in the illuminated region. For example, the PPG signal may indicate blood volume changes in the illuminated region caused by a user's pulse pressure. The processing module 230-a may sample the PPG signal and determine a user's pulse waveform based on the PPG signal. The processing module 230-a may determine a variety of physiological parameters based on the user's pulse waveform, such as a user's respiratory rate, heart rate, HRV, oxygen saturation, and other circulatory parameters.

In some implementations, the PPG system 235 may be configured as a reflective PPG system 235 where the optical receiver(s) receive transmitted light that is reflected through the region of the user's finger. In some implementations, the PPG system 235 may be configured as a transmissive PPG system 235 where the optical transmitter(s) and optical receiver(s) are arranged opposite to one another, such that light is transmitted directly through a portion of the user's finger to the optical receiver(s).

The number and ratio of transmitters and receivers included in the PPG system 235 may vary. Example optical transmitters may include LEDs. The optical transmitters may transmit light in the infrared spectrum and/or other spectrums. Example optical receivers may include, but are not limited to, photosensors, phototransistors, and photodiodes. The optical receivers may be configured to generate PPG signals in response to the wavelengths received from the optical transmitters. The location of the transmitters and receivers may vary. Additionally, a single device may include reflective and/or transmissive PPG systems 235.

The PPG system 235 illustrated in FIG. 2 may include a reflective PPG system 235 in some implementations. In these implementations, the PPG system 235 may include a centrally located optical receiver (e.g., at the bottom of the ring 104) and two optical transmitters located on each side of the optical receiver. In this implementation, the PPG system 235 (e.g., optical receiver) may generate the PPG signal based on light received from one or both of the optical transmitters. In other implementations, other placements, combinations, and/or configurations of one or more optical transmitters and/or optical receivers are contemplated.

The processing module 230-a may control one or both of the optical transmitters to transmit light while sampling the PPG signal generated by the optical receiver. In some implementations, the processing module 230-a may cause the optical transmitter with the stronger received signal to transmit light while sampling the PPG signal generated by the optical receiver. For example, the selected optical transmitter may continuously emit light while the PPG signal is sampled at a sampling rate (e.g., 250 Hz).

Sampling the PPG signal generated by the PPG system 235 may result in a pulse waveform that may be referred to as a “PPG.” The pulse waveform may indicate blood pressure vs time for multiple cardiac cycles. The pulse waveform may include peaks that indicate cardiac cycles. Additionally, the pulse waveform may include respiratory induced variations that may be used to determine respiration rate. The processing module 230-a may store the pulse waveform in memory 215 in some implementations. The processing module 230-a may process the pulse waveform as it is generated and/or from memory 215 to determine user physiological parameters described herein.

The processing module 230-a may determine the user's heart rate based on the pulse waveform. For example, the processing module 230-a may determine heart rate (e.g., in beats per minute) based on the time between peaks in the pulse waveform. The time between peaks may be referred to as an interbeat interval (IBI). The processing module 230-a may store the determined heart rate values and IBI values in memory 215.

The processing module 230-a may determine HRV over time. For example, the processing module 230-a may determine HRV based on the variation in the IBIs. The processing module 230-a may store the HRV values over time in the memory 215. Moreover, the processing module 230-a may determine the user's respiratory rate over time. For example, the processing module 230-a may determine respiratory rate based on frequency modulation, amplitude modulation, or baseline modulation of the user's IBI values over a period of time. Respiratory rate may be calculated in breaths per minute or as another breathing rate (e.g., breaths per 30 seconds). The processing module 230-a may store user respiratory rate values over time in the memory 215.

The ring 104 may include one or more motion sensors 245, such as one or more accelerometers (e.g., 6-D accelerometers) and/or one or more gyroscopes (gyros). The motion sensors 245 may generate motion signals that indicate motion of the sensors. For example, the ring 104 may include one or more accelerometers that generate acceleration signals that indicate acceleration of the accelerometers. As another example, the ring 104 may include one or more gyro sensors that generate gyro signals that indicate angular motion (e.g., angular velocity) and/or changes in orientation. The motion sensors 245 may be included in one or more sensor packages. An example accelerometer/gyro sensor is a Bosch BM1160 inertial micro electro-mechanical system (MEMS) sensor that may measure angular rates and accelerations in three perpendicular axes.

The processing module 230-a may sample the motion signals at a sampling rate (e.g., 50 Hz) and determine the motion of the ring 104 based on the sampled motion signals. For example, the processing module 230-a may sample acceleration signals to determine acceleration of the ring 104. As another example, the processing module 230-a may sample a gyro signal to determine angular motion. In some implementations, the processing module 230-a may store motion data in memory 215. Motion data may include sampled motion data as well as motion data that is calculated based on the sampled motion signals (e.g., acceleration and angular values).

The ring 104 may store a variety of data described herein. For example, the ring 104 may store temperature data, such as raw sampled temperature data and calculated temperature data (e.g., average temperatures). As another example, the ring 104 may store PPG signal data, such as pulse waveforms and data calculated based on the pulse waveforms (e.g., heart rate values, IBI values, HRV values, and respiratory rate values). The ring 104 may also store motion data, such as sampled motion data that indicates linear and angular motion.

The ring 104, or other computing device, may calculate and store additional values based on the sampled/calculated physiological data. For example, the processing module 230 may calculate and store various metrics, such as sleep metrics (e.g., a Sleep Score), activity metrics, and readiness metrics. In some implementations, additional values/metrics may be referred to as “derived values.” The ring 104, or other computing/wearable device, may calculate a variety of values/metrics with respect to motion. Example derived values for motion data may include, but are not limited to, motion count values, regularity values, intensity values, metabolic equivalence of task values (METs), and orientation values. Motion counts, regularity values, intensity values, and METs may indicate an amount of user motion (e.g., velocity/acceleration) over time. Orientation values may indicate how the ring 104 is oriented on the user's finger and if the ring 104 is worn on the left hand or right hand.

In some implementations, motion counts and regularity values may be determined by counting a number of acceleration peaks within one or more periods of time (e.g., one or more 30 second to 1 minute periods). Intensity values may indicate a number of movements and the associated intensity (e.g., acceleration values) of the movements. The intensity values may be categorized as low, medium, and high, depending on associated threshold acceleration values. METs may be determined based on the intensity of movements during a period of time (e.g., 30 seconds), the regularity/irregularity of the movements, and the number of movements associated with the different intensities.

In some implementations, the processing module 230-a may compress the data stored in memory 215. For example, the processing module 230-a may delete sampled data after making calculations based on the sampled data. As another example, the processing module 230-a may average data over longer periods of time in order to reduce the number of stored values. In a specific example, if average temperatures for a user over one minute are stored in memory 215, the processing module 230-a may calculate average temperatures over a five minute time period for storage, and then subsequently erase the one minute average temperature data. The processing module 230-a may compress data based on a variety of factors, such as the total amount of used/available memory 215 and/or an elapsed time since the ring 104 last transmitted the data to the user device 106.

Although a user's physiological parameters may be measured by sensors included on a ring 104, other devices may measure a user's physiological parameters. For example, although a user's temperature may be measured by a temperature sensor 240 included in a ring 104, other devices may measure a user's temperature. In some examples, other wearable devices (e.g., wrist devices) may include sensors that measure user physiological parameters. Additionally, medical devices, such as external medical devices (e.g., wearable medical devices) and/or implantable medical devices, may measure a user's physiological parameters. One or more sensors on any type of computing device may be used to implement the techniques described herein.

The physiological measurements may be taken continuously throughout the day and/or night. In some implementations, the physiological measurements may be taken during portions of the day and/or portions of the night. In some implementations, the physiological measurements may be taken in response to determining that the user is in a specific state, such as an active state, resting state, and/or a sleeping state. For example, the ring 104 can make physiological measurements in a resting/sleep state in order to acquire cleaner physiological signals. In one example, the ring 104 or other device/system may detect when a user is resting and/or sleeping and acquire physiological parameters (e.g., temperature) for that detected state. The devices/systems may use the resting/sleep physiological data and/or other data when the user is in other states in order to implement the techniques of the present disclosure.

In some implementations, as described previously herein, the ring 104 may be configured to collect, store, and/or process data, and may transfer any of the data described herein to the user device 106 for storage and/or processing. In some aspects, the user device 106 includes a wearable application 250, an operating system (OS), a web browser application (e.g., web browser 280), one or more additional applications, and a GUI 275. The user device 106 may further include other modules and components, including sensors, audio devices, haptic feedback devices, and the like. The wearable application 250 may include an example of an application (e.g., “app”) that may be installed on the user device 106. The wearable application 250 may be configured to acquire data from the ring 104, store the acquired data, and process the acquired data as described herein. For example, the wearable application 250 may include a user interface (UI) module 255, an acquisition module 260, a processing module 230-b, a communication module 220-b, and a storage module (e.g., database 265) configured to store application data.

In some cases, the wearable device 104 and the user device 106 may be included within (or make up) the same device. For example, in some cases, the wearable device 104 may be configured to execute the wearable application 250, and may be configured to display data via the GUI 275.

The various data processing operations described herein may be performed by the ring 104, the user device 106, the servers 110, or any combination thereof. For example, in some cases, data collected by the ring 104 may be pre-processed and transmitted to the user device 106. In this example, the user device 106 may perform some data processing operations on the received data, may transmit the data to the servers 110 for data processing, or both. For instance, in some cases, the user device 106 may perform processing operations that require relatively low processing power and/or operations that require a relatively low latency, whereas the user device 106 may transmit the data to the servers 110 for processing operations that require relatively high processing power and/or operations that may allow relatively higher latency.

In some aspects, the ring 104, user device 106, and server 110 of the system 200 may be configured to evaluate sleep patterns for a user. In particular, the respective components of the system 200 may be used to collect data from a user via the ring 104, and generate one or more scores (e.g., Sleep Score, Readiness Score) for the user based on the collected data. For example, as noted previously herein, the ring 104 of the system 200 may be worn by a user to collect data from the user, including temperature, heart rate, HRV, and the like. Data collected by the ring 104 may be used to determine when the user is asleep in order to evaluate the user's sleep for a given “sleep day.” In some aspects, scores may be calculated for the user for each respective sleep day, such that a first sleep day is associated with a first set of scores, and a second sleep day is associated with a second set of scores. Scores may be calculated for each respective sleep day based on data collected by the ring 104 during the respective sleep day. Scores may include, but are not limited to, Sleep Scores, Readiness Scores, and the like.

In some cases, “sleep days” may align with the traditional calendar days, such that a given sleep day runs from midnight to midnight of the respective calendar day. In other cases, sleep days may be offset relative to calendar days. For example, sleep days may run from 6:00 pm (18:00) of a calendar day until 6:00 pm (18:00) of the subsequent calendar day. In this example, 6:00 pm may serve as a “cut-off time,” where data collected from the user before 6:00 pm is counted for the current sleep day, and data collected from the user after 6:00 pm is counted for the subsequent sleep day. Due to the fact that most individuals sleep the most at night, offsetting sleep days relative to calendar days may enable the system 200 to evaluate sleep patterns for users in such a manner that is consistent with their sleep schedules. In some cases, users may be able to selectively adjust (e.g., via the GUI) a timing of sleep days relative to calendar days so that the sleep days are aligned with the duration of time that the respective users typically sleep.

In some implementations, each overall score for a user for each respective day (e.g., Sleep Score, Readiness Score) may be determined/calculated based on one or more “contributors,” “factors,” or “contributing factors.” For example, a user's overall Sleep Score may be calculated based on a set of contributors, including: total sleep, efficiency, restfulness, REM sleep, deep sleep, latency, timing, or any combination thereof. The Sleep Score may include any quantity of contributors. The “total sleep” contributor may refer to the sum of all sleep periods of the sleep day. The “efficiency” contributor may reflect the percentage of time spent asleep compared to time spent awake while in bed, and may be calculated using the efficiency average of long sleep periods (e.g., primary sleep period) of the sleep day, weighted by a duration of each sleep period. The “restfulness” contributor may indicate how restful the user's sleep is, and may be calculated using the average of all sleep periods of the sleep day, weighted by a duration of each period. The restfulness contributor may be based on a “wake up count” (e.g., sum of all the wake-ups (when user wakes up) detected during different sleep periods), excessive movement, and a “got up count” (e.g., sum of all the got-ups (when user gets out of bed) detected during the different sleep periods).

The “REM sleep” contributor may refer to a sum total of REM sleep durations across all sleep periods of the sleep day including REM sleep. Similarly, the “deep sleep” contributor may refer to a sum total of deep sleep durations across all sleep periods of the sleep day including deep sleep. The “latency” contributor may signify how long (e.g., average, median, longest) the user takes to go to sleep, and may be calculated using the average of long sleep periods throughout the sleep day, weighted by a duration of each period and the number of such periods (e.g., consolidation of a given sleep stage or sleep stages may be its own contributor or weight other contributors). Lastly, the “timing” contributor may refer to a relative timing of sleep periods within the sleep day and/or calendar day, and may be calculated using the average of all sleep periods of the sleep day, weighted by a duration of each period.

By way of another example, a user's overall Readiness Score may be calculated based on a set of contributors, including: sleep, sleep balance, heart rate, HRV balance, recovery index, temperature, activity, activity balance, or any combination thereof. The Readiness Score may include any quantity of contributors. The “sleep” contributor may refer to the combined Sleep Score of all sleep periods within the sleep day. The “sleep balance” contributor may refer to a cumulative duration of all sleep periods within the sleep day. In particular, sleep balance may indicate to a user whether the sleep that the user has been getting over some duration of time (e.g., the past two weeks) is in balance with the user's needs. Typically, adults need 7-9 hours of sleep a night to stay healthy, alert, and to perform at their best both mentally and physically. However, it is normal to have an occasional night of bad sleep, so the sleep balance contributor takes into account long-term sleep patterns to determine whether each user's sleep needs are being met. The “resting heart rate” contributor may indicate a lowest heart rate from the longest sleep period of the sleep day (e.g., primary sleep period) and/or the lowest heart rate from naps occurring after the primary sleep period.

Continuing with reference to the “contributors” (e.g., factors, contributing factors) of the Readiness Score, the “HRV balance” contributor may indicate a highest HRV average from the primary sleep period and the naps happening after the primary sleep period. The HRV balance contributor may help users keep track of their recovery status by comparing their HRV trend over a first time period (e.g., two weeks) to an average HRV over some second, longer time period (e.g., three months). The “recovery index” contributor may be calculated based on the longest sleep period. Recovery index measures how long it takes for a user's resting heart rate to stabilize during the night. A sign of a very good recovery is that the user's resting heart rate stabilizes during the first half of the night, at least six hours before the user wakes up, leaving the body time to recover for the next day. The “body temperature” contributor may be calculated based on the longest sleep period (e.g., primary sleep period) or based on a nap happening after the longest sleep period if the user's highest temperature during the nap is at least 0.5° C. higher than the highest temperature during the longest period. In some aspects, the ring may measure a user's body temperature while the user is asleep, and the system 200 may display the user's average temperature relative to the user's baseline temperature. If a user's body temperature is outside of their normal range (e.g., clearly above or below 0.0), the body temperature contributor may be highlighted (e.g., go to a “Pay attention” state) or otherwise generate an alert for the user.

In some aspects, the wearable devices 104 of the system 200 may include multiple differently-sized protrusions that include light-emitting and/or light-receiving components used to acquire physiological data from users 102. The differently-sized protrusions may exhibit varying contact pressures with the user's tissue, where a difference in contact pressures between the protrusions remains constant. In some aspects, the system 200 may support techniques for the wearable device 104 to determine whether changes in PPG signals are due to pressure effects (e.g., due to a finger swelling, aging, ring tightness, or some other pressure effect), or due to actual biological changes of a user. Additionally, or alternatively, the wearable device 104 may determine which LED or PD (e.g., in the PPG system 235) may be associated with a highest quality of measurement data, and may select to use LEDs or PDs within specific dome-shaped protrusions (e.g., at the determined contact pressure) to perform the measurements.

For example, the inner housing 205-a of the wearable device 104 (e.g., a ring 104 or another wearable device 104 such as a wrist-worn wearable device) may include two or more protrusions extending varying distances from the internal surface of the ring 104. The PPG system 235 may include one or more light emitting components (e.g., LEDs) and/or light receiving components (e.g., PDs) disposed within each of the two or more protrusions. Each of the one or more protrusions may contact a surface of tissue of the user at a different contact pressure such that a difference in contact pressures between each of the two or more protrusions is constant (or relatively/substantially constant). The wearable device 104 may determine a quality of measurements or may select one or more LEDs and/or PDs to perform measurements based on a difference in measurement or signal quality (e.g., based on the constant pressure difference). That is, the wearable device 104 may evaluate light-emitting components and/or PDs disposed within each of the differently-sized protrusions to determine which combinations of LEDs and PDs should be used to perform physiological measurements.

In some examples, the wearable device 104 may autonomously determine which combination of LEDs/PDs should be used for data collection (e.g., via the processing module 230-a). In some examples, the wearable device 104 may transmit data collected via sensors disposed within each of the protrusions to the user device 106 and/or servers 110 (e.g., via the communication modules 220-a and 220-b), where the user device 106 and/or servers 110 may perform the determination (e.g., via the processing module 230-b). The user device 106 may transmit an instruction to the wearable device 104 (e.g., via the communication modules 220-a and 220-b) to select the LEDs and/or PDs to perform the measurements. Accordingly, by evaluating sensors within differently-sized protrusions, the wearable device 104 may collect more accurate measurement data as compared to wearable devices 104 that do not include multiple differently-sized protrusions, which may result in more accurate physiological data and higher user satisfaction.

In some examples, differently-sized protrusions may include light-emitting components (e.g., LEDs, laser diodes, etc.), light-receiving components (e.g., PDs), or both. In other cases, the differently-sized protrusions may include light-emitting components, where the PDs may be located in a different position along the wearable device 104 (e.g., outside of the protrusions). In yet other cases, the differently-sized protrusions may include light-receiving components, where the light-emitting components may be located in a different position along the wearable device 104 (e.g., outside of the protrusions).

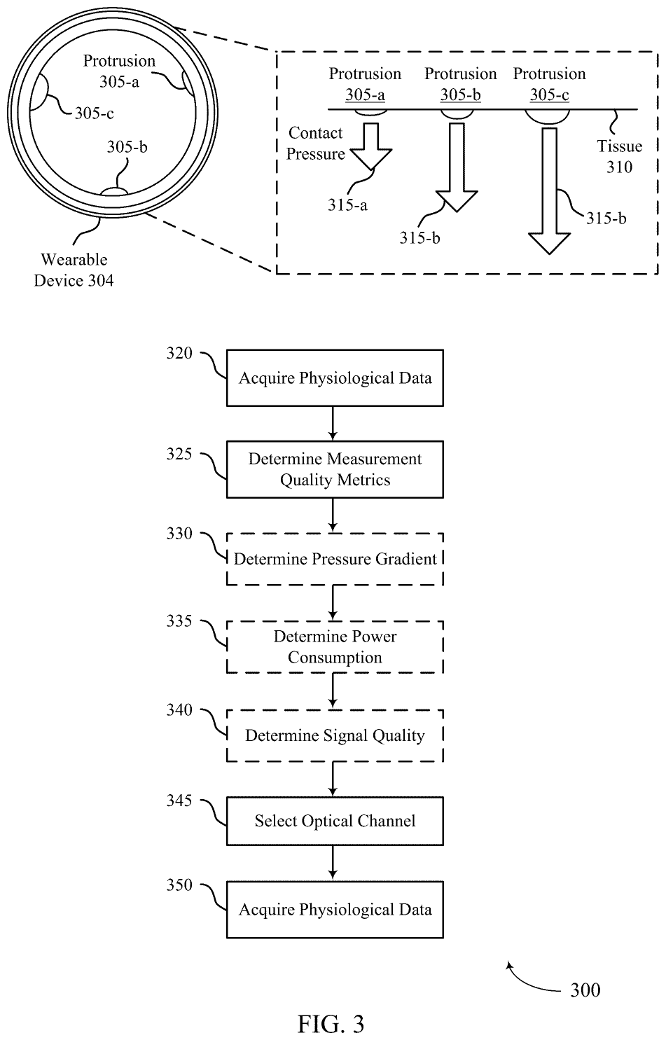

FIG. 3 shows an example of a process flow 300 that supports techniques for leveraging stable pressure differences for optical measurements using a wearable device in accordance with aspects of the present disclosure. The process flow 300 may implement, or be implemented by, system 100, system 200, or both. In particular, the process flow 300 illustrates an example of a wearable device 304 (e.g., a wearable device 104), as described with reference to FIGS. 1 and 2.

In some examples, the wearable device 304 (e.g., a wearable ring device, a wrist-based wearable device) may include a housing with an internal surface and an external surface, which may be examples of an inner housing 205-a and an outer housing 205-b as described with reference to FIG. 2. The internal surface of the wearable device 304 may at least partially contact a tissue 310 of a user and may include one or more protrusions 305 that the wearable device 304 may use to obtain measurements of physiological data of the user (e.g., PPG measurements to detect a blood oxygen saturation level, blood pressure, cardiovascular age, and so on of the user).

For example, the one or more protrusions 305 may each include one or more optical components (e.g., light-emitting components such as one or more LEDs disposed within the protrusions 305 and/or light-receiving components such as one or more PDs). As noted previously herein, each protrusion 305 may include light-emitting component(s), light-receiving component(s), or both. In other cases, the protrusions 305 may include light-emitting components, where the light-receiving components are located separately from the protrusions 305 (or vice versa). The wearable device 304 may include one or more LEDs (e.g., disposed within a first protrusion 305), which may emit light received by the one or more PDs. In this regard, each LED may support one or more optical channels for physiological data measurements. For the purposes of the present disclosure, the term “optical channel” may refer to a measurement channel for collecting physiological data that includes one light-emitting component and one light-receiving component. In this regard, different combinations of LEDs and PDs may be used to acquire data along different optical channels, where a single LED or PD may be used in one or more optical channels. The wearable device may include any number of LEDs, PDs, and respective optical channels for physiological data measurements (e.g., LEDs and PDs disposed within protrusions 305). In some examples, the protrusions 305 may be made of an optically clear epoxy material such that light from the LEDs may move through the protrusions 305 into tissue 310 of the user of the wearable device 304.

In some examples, the one or more light-emitting components (e.g., LEDs, laser diodes, etc.) may be configured to transmit light of varying wavelengths (e.g., varying colors) to enable the wearable device 304 to propagate multiple light waves into the tissue 310 at varying measurement depths. That is, the penetration depth (e.g., wavelength range) of light into the tissue 310 may increase with wavelength from the UV to the visible light range and through the IR range. In other words, green light and red light transmitted by the same (or different) light-emitting component may penetrate the tissue 310 of the user to different penetration depths.

For example, the light-emitting and light-receiving components of the wearable device 304 may be coupled to a controller to transmit and receive light associated with one or more wavelengths. As an illustrative example, the controller of the wearable device 304 may cause an LED transmit a blue light associated with a wavelength of approximately 460 nanometers (nm), a green light associated with a wavelength of approximately 530 nm, a red light associated with a wavelength of approximately 660 nm, and/or an IR light associated with a wavelength of approximately 940 nm, where each of the transmitted wavelengths of light may reach one or more layers of tissue (e.g., an epidermis layer at around 0.3 millimeters (mm), a dermis layer at around 1.0 mm, a hypodermis layer at around 3.0 mm). That is, each of the transmitted lights may reach the one or more layers of tissues where the blood vessels are located, such as the capillaries located closest to the surface of the tissue 310 at the epidermis, the arterioles located at the middle layer at the dermis, and the arteries located at the deepest layer at the hypodermis. As such, the wearable device 304 may use the light-emitting components and the light-receiving components coupled to a controller on the wearable device 304 to transmit one or more lights associated with wavelength ranges to one or more tissue layers at multiple locations (e.g., multiple penetration depths) to acquire physiological data.

In this regard, the wavelength/color of light affects the penetration depth of the light. Additionally, the penetration depth of light is also affected by the contact pressure 315 between the respective light-emitting component and the tissue 310 of the user. That is, light emitted by the one or more LEDs may penetrate tissue 310 of the user differently depending on a contact pressure 315 applied to a measurement or source area. In other words, green light (or any other wavelength) may penetrate the tissue 310 of the user to different penetration depths depending on the contact pressure 315 between the light-emitting component and the tissue 310 of the user. In particular, the optical interface between sensors (e.g., LEDs, PDs) and the tissue 310 of the user may change with varying contact pressure 315, where the varying contact pressure affects how well light travels across the optical interface. In general, higher contact pressure may result in a more efficient optical interface, and increased penetration depths for a given wavelength of light. For example, a relatively influential/efficient optical interface type may be created between the protrusions 305 and the tissue 310 with good skin contact and embedded LEDs. A total internal reflection (TIR) critical angle may be relatively large over an optical interface (e.g., 81 degrees), and light out-coupling from the inner housing may be relatively efficient (e.g., <0.1% light lost at the interface via Fresnel reflections).

The actual physiology of the user's tissue is another important mechanism that links optical sensor contact pressure to increased light transmission. When contact pressure between an LED (e.g., LED beneath/within a protrusion 305) and the user's tissue 310 increases, blood is pushed away from upper layers of the user's tissue 310 around the contact location. As blood is the major light-absorbing bio-material in the skin tissue 310, this increases light transmission through the skin layers (e.g., higher contact pressure results in less blood to absorb light, thereby resulting in increased light transmission). Comparatively, when there is less (or no) contact pressure between the LED and the user's tissue 310 (but where the LED/sensor is still touching the tissue 310), the received PPG signals come from all skin layers. As the contact pressure is increased, the top/upper skin layers start to become more transparent or translucent (due to the increased contact pressure pushing blood away within the upper skin layers), while lower/deeper layers of skin/tissue 310 are less affected. In such cases, the PPG alternating current (AC) signal becomes stronger as light penetrates deeper into the tissue (as upper layers of tissue become more transparent/translucent) and gets absorbed more by the larger blood vessels. If the contact pressure is increased to a high level, the high contact pressure may start to act against the internal pressure of the larger blood vessels, and can eventually block blood flow, resulting in PPG signals with lower quality/strength.

Taken together, there may be an optimal sensor contact pressure somewhere between “no-pressure” and high-pressure that results in the highest quality PPG signals. As the external contact pressure (e.g., contact pressure between sensors of a wearable and the user's tissue 310) acts against blood vessel internal pressure, there may be a correlation between sensor contact pressure and blood pressure that could be used for measuring blood pressure.

However, in some cases, skin contact (e.g., contact pressure 315) between the tissue 310 and the wearable device 304 may vary due to an external force pushing on the wearable device 304, a gap between the tissue 310 and the wearable device 304, liquid or contaminants between the tissue 310 and the wearable device 304, aging of the user, swelling of the tissue 310 (e.g., hydration, dehydration), or a combination thereof. Changes in skin contact or contact pressure 315 against the tissue 310 may affect light penetration depths and physiological measurements and may change over time due to physiological differences in the user (e.g., swelling, age, and the like). A change or difference in contact pressure 315 between the protrusions 305 and the tissue 310 may result in variations of light signals, penetration depths, signal paths, the TIR and so on, which may cause losses to light coupled from LED optics and therefore relatively lower measurement quality.

Accordingly, techniques described herein may allow the wearable device 304 to identify a fixed pressure difference between protrusions 305, which may create more stable signals relative to changes in contact pressure 315. For example, the internal surface of the wearable device 304 may include at least a protrusion 305-a and a protrusion 305-b extending from the internal surface and resulting in a contact pressure 315-a and a contact pressure 315-b with the tissue 310 of the user, respectively. In some examples, the protrusion 305-a may be smaller (e.g., shorter) than the protrusion 305-b. That is, the protrusion 305-a may extend a first distance from the internal surface, and the protrusion 305-b may extend a second distance from the internal surface (e.g., greater than the first distance). In some cases, the wearable device 304 may also include one or more additional protrusions 305, such as a protrusion 305-c extending a third distance (e.g., greater than the second distance) from the internal surface and resulting in a contact pressure 315-c with the tissue 310.

In some examples, the protrusion 305-a, the protrusion 305-b, and the protrusion 305-c may be located in different radial positions within the wearable device 304. For example, in cases where the wearable device 304 is a ring, as shown in FIG. 3, the first protrusion 305-a may be located at a first radial position along an inner curved surface (e.g., inner circumferential surface) of the wearable device 304 and the second protrusion 305-b may be located at a second radial position along the inner curved surface. One or more additional protrusions (e.g., the third protrusion 305-c) may be located at one or more additional radial positions along the internal curved surface. The protrusions 305 may be evenly distributed along the inner curved surface (e.g., to avoid compression of capillaries from clustered protrusions 305). Additionally, or alternatively, the protrusions 305 may be clustered such that a curved distance (e.g., radial distance, circumferential distance) between one or more of the first location, the second location, and the one or more additional locations may be different.

For the purposes of the present disclosure, the term “curved,” “circumferential,” and like terms, may be used interchangeably to refer to any surface or shape that exhibits a curved profile or contour. As such, the terms “curved” and “circumferential,” may be used to refer to surfaces/shapes that are circular, elliptical, etc., unless noted otherwise herein. Similarly, the term “circumference” may be used to refer to the shape of a wearable ring device that wraps radially around a user's finger (e.g., 360° perimeter or shape), and is not to be interpreted as referring solely to a perfectly circular or elliptical shape. That is, the term “circumference” may be used to refer to any radial span that extends radially (e.g.,) 360° around the ring. For example, a wearable ring device may be said to have a “circumference” that wraps radially around the user's finger even in cases where the ring itself is not a perfect circle or ellipse. That is, wearable ring devices with an elliptical shape, flat portions, etc. may still be said to exhibit a “circumference” in that the wearable ring devices exhibit a shape/perimeter that wraps around a user's finger.

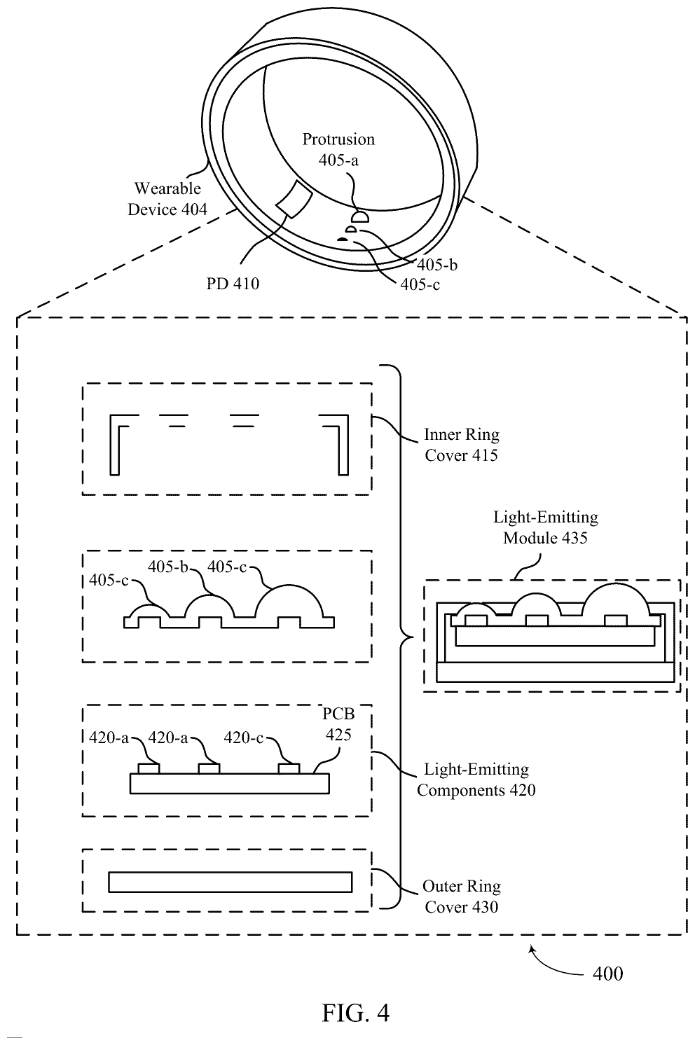

In some examples, the protrusion 305-a, the protrusion 305-b, and the protrusion 305-c may be in one or more other locations along the inner curved surface of the wearable device 304. For example, the protrusion 305-a, the protrusion 305-b, and the protrusion 305-c may be in a same (or similar) radial position along the inner curved surface of the wearable device 304 and in different positions along a width of the inner curved surface of the wearable device 304. Such a configuration is described in further detail with reference to FIG. 4. In some examples, one or more of the protrusion 305-a, the protrusion 305-b, and the protrusion 305-c may be a same size. For example, the protrusion 305-a and the protrusion 305-c may extend a same height above the inner curved surface of the wearable device 304, and the protrusion 305-b may extend a different height above the inner curved surface (e.g., smaller than or larger than the first height).

In some examples, the light-emitting components (e.g., LEDs, laser diodes) may be disposed within each of the protrusions 305. Additionally, or alternatively, the wearable device 304 may include a single laser diode or VCSEL optical components. In such examples, the wearable device may include a lightguide that may branch light emissions from the laser diode or VCSEL to each of the protrusions 305. The wearable device 304 may also include an electro-optical switch that may select an optical channel of the wearable device 304 to use. In other words, a single light source may be used (in conjunction with a switch) to provide light to each of the protrusions (or other locations for emitting light into the tissue 310 of the user). Such techniques may result in a relatively more stable light output (e.g., in terms of light intensity and spectrum) than separate LEDs at each of the protrusions 305. Additionally, a VCSEL may emit a relatively more narrow beam of light as compared to an LED. Accordingly, the VCSEL may result in relatively less signal variation during movement (e.g., due to a relatively smaller area of the tissue 310 that may be in contact with the dome to receive the signal).

In some cases, the protrusions 305 may additionally, or alternatively, include light-receiving components (e.g., PDs). In some examples, a light-receiving component may be located at a third position along the inner curved surface (e.g., between the first and second radial positions of the protrusions 305). In such examples, a distance between the third position and the first position (e.g., a first light-emitting component disposed within the protrusion 305-a) and a distance between the third position and the second position (e.g., a second light-emitting component disposed within the protrusion 305-b) may be equal. Stated differently, a PD may be located along the inner surface of the wearable device 304 such that a first distance between the PD and the first LED in the first protrusion 305-a and a second distance between the PD and the second LED in the second protrusion 305-b is the same. In such examples, the wearable device may use a first optical channel between the first LED and the PD and a second optical channel between the second LED and the PD to perform measurements of physiological data (e.g., PPG measurements). The wearable device 304 may additionally, or alternatively, use one or more additional optical channels between the first light-emitting component, the second light-emitting component, one or more additional light-emitting components (e.g., disposed within the protrusion 305-c or one or more additional protrusions 305), the light-receiving component, and/or one or more additional light-receiving components. For example, one or more light-receiving components (e.g., PDs) may additionally, or alternatively, be disposed within each of the protrusions 305.

In some examples, the first light-emitting component and the second light-emitting component (e.g., and the one or more additional light-emitting components) may emit a first wavelength of light, a second wavelength of light, or both (e.g., to penetrate the tissue 310 to different penetration depths based on the respective wavelengths and contact pressures 315). For example, the green light and red light emitted from an LED within a same protrusion 305 may penetrate the tissue 310 to different penetration depths based on the different wavelengths. Moreover, green light emitted from LEDs within the first protrusion 305-a and the second protrusion 305-b may penetrate the tissue 310 to different penetration depths based on the varying contact pressures 315 at the respective protrusions 305.

In some examples, each protrusion 305 may also include a light-blocking element. For example, the first protrusion 305-a may include a first light-blocking element extending a first height from the internal surface of the housing, and the second protrusion 305-b may comprise a second light-blocking element extending a second height from the internal surface of the housing. Light-blocking elements disposed along the surfaces of the protrusions may ensure that light transmitted and/or received by optical elements (e.g., light-transmitting and light-receiving components) is transmitted/received along specific angles or optical paths. For example, a light-blocking element disposed along lateral surfaces of the third protrusion 305-c may ensure that an LED disposed within the protrusion 305-c emits light substantially perpendicular to the tissue 310/surface of the wearable device 304.

At 320, the wearable device 304 may use one or more processors to acquire physiological data from the user using at least a first optical channel and a second optical channel (e.g., via optical components disposed within the protrusion 305-a and the protrusion 305-b, such as the first light-emitting component and the second light-emitting component, and one or more light-receiving components). For example, the wearable device 304 may transmit first light and second light associated with the first wavelength via the first light-emitting component disposed within the protrusion 305-a and the second light-emitting component disposed within the protrusion 305-b, respectively. The wearable device 304 may additionally transmit third light of the first wavelength via a third light-emitting component disposed within the protrusion 305-c, and one or more additional lights via light-emitting components in one or more additional protrusions 305. In other words, the wearable device 304 may collect physiological data using green light (or some other wavelength) by leveraging optical components disposed within the respective protrusions 305. The wearable device 304 may acquire the physiological data during a first time interval.

In some examples, the wearable device 304 may acquire additional physiological data by transmitting light associated with the second wavelength (e.g., and one or more additional wavelengths) via the first light-emitting component and the second light-emitting component. In some examples, the wearable device 304 may receive the first light and the second light via the one or more light-receiving components. In other words, the wearable device 304 may acquire additional physiological data using a different wavelength (e.g., green light, IR light) and leveraging the optical components disposed within the respective protrusions 305.

At 325, the wearable device 304 may determine one or more measurement quality metrics via the one or more processors based on receiving the first light and the second light. That is, the wearable device 304 may determine a first measurement quality associated with the first optical channel associated with optical component(s) within the first protrusion 305-a, and a second measurement quality associated with the second optical channel associated with optical component(s) within the second protrusion 305-b. The first and second measurement qualities may be associated with measurements of physiological data of the user. In some examples, measurement quality metrics associated with the first optical channel and the second optical channel may be associated with the contact pressure 315-a and the contact pressure 315-b, respectively. The measurement quality metrics may include signal strength metrics or other metrics indicative of signal quality.

In some examples, at 330, the wearable device 304 may determine a pressure gradient associated with the tissue of the user based on receiving the first light and the second light (e.g., using the one or more processors). That is, by transmitting the same wavelength of light from different protrusions 305 (and therefore different contact pressures), the wearable device 304 may be able to determine a pressure gradient of the tissue 310. For example, the wearable device 304 may determine a pressure gradient between the contact pressure 315-a associated with the protrusion 305-a in the first radial position and the contact pressure 315-b associated with the protrusion 305-b in the second radial position (e.g., and one or more other contact pressures 315 associated with one or more protrusions 305, such as the protrusion 305-c). For instance, by transmitting green light from LEDs within the first protrusion 305-a and the second protrusion 305-b, and measuring the signal strength of the green light received at one or more PDs, the wearable device 304 (or another device) may be able to determine how the green light is absorbed by the tissue 301 at different contact pressures 315, and therefore at different penetration depths. As such, this information may be used to create a “pressure gradient” that indicates how different layers of tissue 310 are compressed, and/or how different layers of tissue absorb light.

In some cases, a determined pressure gradient may be used to determine, calibrate, or improve physiological measurements of the user. The wearable device 304 may determine one or more physiological parameters (e.g., blood pressure, cardiovascular age) based on the pressure gradient (e.g., and based on the measurement qualities). For example, the wearable device 304 may determine whether one or more physiological parameters of the user have changed or if one or more measurements via the first optical channel and the second optical channel have changed due to the pressure gradient. For instance, based on the pressure gradient, the wearable device 304 (or another device) may be able to estimate the penetration depth of the light and/or estimate how much light has been absorbed by the tissue 310 due to compression of the various layers of tissue 310. Such calculations/estimations may be used to calibrate or otherwise adjust physiological measurements. This approach may eliminate the need for separate active pressure measurements and thus enable improved blood pressure measurements and also measurements of physiological changes in skin capillaries, for example.

In some examples, the wearable device 304 may determine a metric associated with how well the wearable device 304 may fit on the user (e.g., around a finger or wrist of the user) based on the pressure gradient. For example, the wearable device 304 may determine that the contact pressure 315-b at the second protrusion 305-b is high, and the contact pressure 315-a at the first protrusion 305-a is low (e.g., zero), which may indicate that the ring is large or may indicate hard one-sided contact on the ring (e.g., the user grabbing an object). The wearable device 304 may calibrate data acquisition based on the determination.

In some examples, at 335, the wearable device 304 may determine respective power consumption metrics associated with each of the first optical channel and the second optical channel (e.g., and one or more additional optical channels). In other words, the wearable device 304 may determine how much power is consumed to collect physiological data using optical components disposed within the respective protrusions 305. For example, the wearable device 304 may determine a quantity of power consumed to collect physiological data along using optical components within the first protrusion 305-a associated with the first optical channel, and power consumed to collect physiological data using optical components within the second protrusion 305-b associated with the second optical channel.

In some examples, at 340, the wearable device 304 may determine one or more signal strength or quality metrics associated with the first optical channel including optical components within the first protrusion 305-a, and one or more signal strength or quality metrics associated with the second optical channel including optical components within the second protrusion 305-b. The one or more signal strength or quality metrics may include a strength or quality (e.g., power, amplitude) of an alternating current (AC) or direct current (DC) component of a PPG signal. In some examples, the signal quality metric may include a trade-off between a transmitting signal strength (e.g., a power consumption used to transmit the PPG signal) and a signal quality (e.g., a strength of a received signal). The wearable device 304 may compare a difference in signal strength or quality between the first optical channel and the second optical channel to a difference in signal strength or quality determined during one or more previous time intervals to determine the physiological parameters.

As an illustrative example, in a previous first interval (e.g., Day 1), the wearable device 304 may perform measurements (e.g., PPG measurements) using the first wavelength via the first light-emitting component within the first protrusion 305-a (e.g., via the first optical channel) at a first height to obtain a signal strength or quality metric (PPG_{Day 1,Height1}), and via the second light-emitting component within the second protrusion 305-b (e.g., via the second optical channel) at a second height to obtain a signal strength or quality metric (PPG_{Day 1,Height2}). In such examples, the different height protrusions 305 may result in a difference in signal strength or quality (ΔPPG_{Day 1}) due to differences in the contact pressure 315-a and the contact pressure 315-b (e.g., ΔPPG_{Day 1}=PPG_{Day 1,Height1}−PPG_{Day 1, Height2}). In other words, on Day 1, the wearable device 304 may transmit IR light (or some other wavelength) from different height protrusions 305 to collect physiological data.

During a second time interval (e.g., Day 2), the wearable device 304 may transmit light of the first wavelength (e.g., IR light) via the protrusions 305-a and 305-b to acquire additional PPG measurements (e.g., PPG_{Day 2,Height1}, PPG_{Day 2,Height2}). In other words, on Day 2, the wearable device 304 may again transmit IR light (or some other wavelength) from the different height protrusions 305 to collect physiological data. As before, the different height protrusions 305 may result in a difference in signal strength or quality (ΔPPG_{Day 2}) due to differences in the contact pressure 315-a and the contact pressure 315-b (e.g., ΔPPG_{Day 2}=PPG_{Day 2,Height1}−PPG_{Day 2, Height2}).

Continuing with the same example, the wearable device 304 may expect the different heights of the protrusions 305-a and 305-b to result in a constant pressure difference between the pressure 315-a and the pressure 315-b, and therefore to result in a constant difference in the signal strengths or qualities. As such, if the difference in signal strengths during the first time interval (Day 1) is the same as that during the second time interval (Day 2) (e.g., if ΔPPG_{Day 1}=ΔPPG_{Day 2}), then the wearable device 304 may determine that any changes in the PPG signals (e.g., changes in the physiological measurements) during the second time interval (Day 2) are attributable to biological changes and are not due to pressure effects, such as swollen fingers.

Comparatively, if the difference in signal strengths or qualities during the first time interval (Day 1) is not the same as that during the second time interval (Day 2) (e.g., if ΔPPG_{Day 1}≠ΔPPG_{Day 2}), then the wearable device 304 may determine that at least some of the changes in the measurements during the second time interval (Day 2) are attributable to pressure effects (swollen fingers, user being dehydrated, aging, the user holding an object, etc.). In such cases, the wearable device 304 may quantify pressure effects from respective PPG signals across the different protrusions 305 and modify collected PPG data to account for such pressure effects. That is, the wearable device 304 may estimate one or more contact pressure 315 changes at the protrusion 305-a, the protrusion 305-b, or both, between Day 1 and Day 2 based on the comparison between the first signal strength difference (ΔPPG_{Day 1}) and the second signal strength difference (ΔPPG_{Day 2}).

The wearable device 304 may calibrate physiological data acquired during respective time intervals based on the signal strength or quality difference (e.g., and based on the contact pressure 315 estimation). For example, the wearable device may determine to modify the physiological data acquired on Day 2 to account for signal quality changes resulting from pressure changes (e.g., swelling and the like) on Day 2 relative to Day 1.

At 345, the wearable device 304 may select an optical channel to perform one or more additional physiological measurements. For example, the wearable device 304 may select one of the first optical channel (e.g., optical components within the first protrusion 305-a), the second optical channel (e.g., optical components within the second protrusion 305-b), or one or more additional optical channels based on the determined pressure gradient, power consumption, measurement quality, signal quality, or some combination thereof. In other words, the wearable device 304 (or another device) may select which protrusions 305 and corresponding optical components should be used to acquire physiological data. The wearable device 304 may select an optical channel (e.g., select optical components within respective protrusion(s)) that may have a lowest power consumption, a highest signal or measurement quality, and/or a determined contact pressure 315 within one or more threshold contact pressures 315 among the optical channels of the wearable device 304. As an illustrative example, if the wearable device 304 determines an increase in contact pressure 315, the wearable device 304 may select an optical channel associated with a smallest protrusion (e.g., the protrusion 305-a).

In some aspects, to perform the selecting at 345, the wearable device 304 may test out different combinations of optical components disposed within different combinations of protrusions 305 (e.g., first optical channel including LED within the first protrusion 305 and PD within the second protrusion 305-b, second optical channel including LED within the second protrusion 305-b and a PD located on the surface of the wearable device 304 separate from protrusions 305, etc.).

At 350, the wearable device 304 may acquire additional physiological data (e.g., blood pressure data, cardiovascular age) from the user using the selected optical channel. In other words, if the wearable device 304 selects the first optical channel at 345 (e.g., selects optical component(s) within the first protrusion 305-a), the wearable device may acquire additional physiological data at 350 using the first optical channel (e.g., using the optical component(s) within the first protrusion 305-a). The wearable device 304 may acquire the additional physiological data based on using the selected optical channel. In some examples, the wearable device may acquire the additional physiological data based on the calibration. Accordingly, the wearable device 304 may perform more accurate measurements of physiological data than without the differently-sized protrusions 305.

FIG. 4 shows an example of a sensor diagram 400 that supports techniques for leveraging stable pressure differences for optical measurements using a wearable device in accordance with aspects of the present disclosure. The sensor diagram 400 may implement, or be implemented by, system 100, system 200, the process flow 300, or any combination thereof. In particular, the sensor diagram 400 illustrates an example of a wearable device 404 (e.g., a wearable device 104), as described with reference to FIGS. 1 and 2.

As illustrated with reference to the sensor diagram 400, a wearable device 404 (e.g., a wearable ring device or a wrist-worn wearable device) may include two or more protrusions 405 (e.g., a first protrusion 405-a, a second protrusion 405-b, and a third protrusion 405-c). While the wearable device 404 of FIG. 4 is illustrated as including three protrusions 405, in some examples, the wearable device 404 may include fewer than three protrusions (e.g., two protrusion) or more than three protrusions. The protrusions 405-a, 405-b, and 405-c may each extend a different height above an internal surface of the wearable device 404, as shown in FIG. 4 and described with reference to FIG. 3. Accordingly, the respective protrusions 405 may result in different contact pressures against a tissue of a user (e.g., first protrusion 405-a results in a highest contact pressure, third protrusion 405-c results in a lowest contact pressure, etc.). In some examples, one or more of the protrusion 405-a, the protrusion 405-b, and the protrusion 405-c may be a same size. For example, the protrusion 405-a and the protrusion 405-c may extend a same height above the inner curved surface of the wearable device 404, and the protrusion 405-b may extend a different height above the inner curved surface (e.g., smaller than or larger than the first height).

In some aspects, the protrusions 405 may house or include one or more light-emitting components 420 (e.g., a light-emitting component 420-a, a light emitting component 420-b, and a light-emitting component 420-c housed with the protrusion 405-a, the protrusion 405-b, and the protrusion 405-c, respectively). The light-emitting components 420 may be examples of one or more LEDs, one or more VCSELs, or one or more single laser diodes. The light-emitting components 420 may each emit light of one or more wavelengths (e.g., green light, red light, infrared light, and so on).

The wearable device 404 may include a PD 410 (e.g., common PD 410) that may receive light emitted from each of the light-emitting components 420 to perform measurements of physiological parameters (e.g., blood pressure, cardiovascular age, and so on) of a user of the wearable device 404. In some examples, the wearable device 404 may use a first optical channel between the PD 410 and the first light-emitting component 420-a, a second optical channel between the PD 410 and the second light-emitting component 420-b, and a third optical channel between the PD 410 and the third light-emitting component 420-c to perform the physiological measurements.

As shown in FIG. 4, the respective protrusions 405 may be located on an inner curved surface (e.g., inner circumferential surface) of the wearable device 404. In particular, each of the respective protrusions 405 may be located at a same (or similar) radial position along the inner curved surface of the wearable device 404, where the PD 410 may be located at a different radial position. By positioning the protrusions 405 (and therefore the light-emitting components 420) at the same (or similar) radial position, signals (e.g., PPG signals) measured by the PD 410 may pass through a same (e.g., or similar) portion of tissue of the user via each optical channel. That is, positioning the protrusions 405/light-emitting components 420 may result in substantially similar optical channels, where the primary difference between the optical channels is the height of the respective protrusions 405, and corresponding contact pressures. As such, this arrangement of protrusions/sensors may enable the wearable device 404 to more effectively determine the effect of varying contact pressures on physiological measurements (e.g., by isolating the protrusion 405 height/contact pressure as the changing variable between the respective optical channels). Comparatively, the sensor arrangement shown and described in FIG. 3 (where the protrusions 305 and/or light-emitting components are positioned at different radial positions) may result in light traveling along different optical paths that go through different finger physiological locations, which may lead to reflections from bone or other tissue types that cause differences in PPG signals collected using the respective optical channels.

In some cases, the arrangement of protrusions 405/sensors shown in FIG. 4 may enable the protrusions and corresponding sensors to be manufactured and/or installed as a single light-emitting module 435. For example, as shown in FIG. 4, each of the protrusions 405 (and corresponding light-emitting components 420) may be housed within a portion of a housing of the wearable device 404 between an inner ring cover 415 and an outer ring cover 430. The protrusions 405 may be manufactured using a material (e.g., a clear resin-based material) that may allow light from each of the light-emitting components 420 to pass through the respective protrusions 405. In some examples, the light-emitting components 420 may be powered and/or controlled by a printed wiring board (PWB) or PCB 425 (e.g., housed between the inner ring cover 415 and outer ring cover 430). The inner ring cover 415, the protrusions 405, the light-emitting components 420 (e.g., and the corresponding PCB 425), and the outer ring cover 430 may form a light-emitting module 435, as illustrated with reference to FIG. 4.

In some examples, the wearable device 404 may use some or all aspects of the light-emitting module 435 (e.g., and the PD 410) to perform the operations of the process flow 300. That is, the wearable device 404 may use the differently-sized protrusions 405 in the configuration illustrated in FIG. 4 to perform more accurate measurements of physiological data than without the differently-sized protrusions via the methods described herein.

While the protrusions 405 of the light-emitting module 435 are shown from smallest to largest left to right, this is not to be regarded as a limitation of the present disclosure. For example, in some cases, the largest protrusion 405-c may be positioned between the two other protrusions 405-a, 405-b. Moreover, in some other cases, the wearable device 404 may include multiple protrusions of the same size. In such cases, the same sized protrusions 405 may be positioned at different locations of the wearable device 404 to further increase a diversity of optical channels, and to enable the wearable device 404 to further evaluate the effect of protrusion 405 height/contact pressure on physiological measurements. For example, in some cases, the light-emitting module 435 may include a tallest protrusion 405 (e.g., third protrusion 405-c) in the middle, and two smaller, equivalently-sized protrusions (e.g., second protrusion 405-b) on each side of the middle protrusion 405. In other cases, the light-emitting module 435 may include a smaller protrusion (e.g., first protrusion 405-a or second protrusion 405-b) in the middle, and two larger, equivalently-sized protrusions (e.g., second protrusion 405-b or third protrusion 405-c) on each side of the middle protrusion 405. In some cases, such symmetric arrangements of the protrusions 405 may enable the wearable device 404 to further increase a diversity of optical channels, and enable the wearable device 404 to further evaluate the effect of protrusion 405 height/contact pressure on physiological measurements.

Moreover, in some cases, the wearable device 404 may include multiple light-emitting modules 435. For example, in some cases, the wearable device 404 may include multiple light-emitting modules 435 positioned at different radial positions around the inner curved surface of the wearable device 404, where each light-emitting module 435 includes differently-sized protrusions that house light-emitting components, light-receiving components, or both.

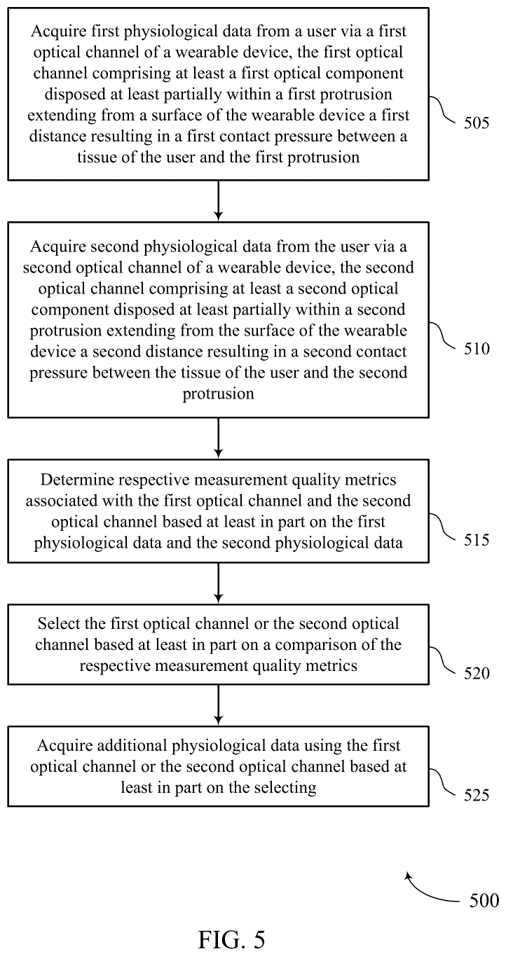

FIG. 5 shows a flowchart illustrating a method 500 that supports techniques for stable pressure difference for optical measurements using a wearable device in accordance with aspects of the present disclosure. The operations of the method 500 may be implemented by a wearable device or its components as described herein. For example, the operations of the method 500 may be performed by a wearable device as described with reference to FIGS. 1-4. In some examples, a wearable device may execute a set of instructions to control the functional elements of the wearable device to perform the described functions. Additionally, or alternatively, the wearable device may perform aspects of the described functions using special-purpose hardware.

At 505, the method may include acquiring first physiological data from a user via a first optical channel of a wearable device, the first optical channel comprising at least a first optical component disposed at least partially within a first protrusion extending from a surface of the wearable device a first distance resulting in a first contact pressure between a tissue of the user and the first protrusion. The operations of 505 may be performed in accordance with examples as disclosed herein.

At 510, the method may include acquiring second physiological data from the user via a second optical channel of a wearable device, the second optical channel comprising at least a second optical component disposed at least partially within a second protrusion extending from the surface of the wearable device a second distance resulting in a second contact pressure between the tissue of the user and the second protrusion. The operations of 510 may be performed in accordance with examples as disclosed herein.

At 515, the method may include determining respective measurement quality metrics associated with the first optical channel and the second optical channel based at least in part on the first physiological data and the second physiological data. The operations of 515 may be performed in accordance with examples as disclosed herein.

At 520, the method may include selecting the first optical channel or the second optical channel based at least in part on a comparison of the respective measurement quality metrics. The operations of 520 may be performed in accordance with examples as disclosed herein.

At 525, the method may include acquiring additional physiological data using the first optical channel or the second optical channel based at least in part on the selecting. The operations of 525 may be performed in accordance with examples as disclosed herein.

The description set forth herein, in connection with the appended drawings, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

In the appended figures, similar components or features may have the same reference label. Further, various components of the same type may be distinguished by following the reference label by a dash and a second label that distinguishes among the similar components. If just the first reference label is used in the specification, the description is applicable to any one of the similar components having the same first reference label irrespective of the second reference label.

Information and signals described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

It should be noted that the methods described above describe possible implementations, and that the operations and the steps may be rearranged or otherwise modified and that other implementations are possible. Furthermore, aspects from two or more of the methods may be combined.

A method by an apparatus is described. The method may include a housing comprising an external surface and an internal surface, the internal surface configured to at least partially contact a tissue of a user, a first protrusion extending from the internal surface of the housing a first distance resulting in a first contact pressure between the tissue and the first protrusion, a second protrusion extending from the internal surface of the housing a second distance resulting in a second contact pressure between the tissue and the second protrusion, a plurality of optical components comprising one or more light-transmitting components and one or more light-receiving components, the plurality of optical components comprising at least a first optical component disposed within the first protrusion, and at least a second optical component disposed within the second protrusion, one or more processors communicatively coupled with the plurality of optical components, the one or more processors configured to, acquire physiological data from the user via at least a first optical channel including the first optical component, and a second optical channel including the second optical component, determine respective measurement quality metrics associated with the first optical channel and the second optical channel based at least in part on the physiological data, select the first optical channel or the second optical channel based at least in part on a comparison of the respective measurement quality metrics, and acquire additional physiological data using the first optical channel or the second optical channel based at least in part on the selecting.

An apparatus is described. The apparatus may include one or more memories storing processor executable code, and one or more processors coupled with the one or more memories. The one or more processors may individually or collectively be operable to execute the code to cause the apparatus to a housing comprise an external surface and an internal surface, the internal surface configured to at least partially contact a tissue of a user, a first protrusion extend from the internal surface of the housing a first distance resulting in a first contact pressure between the tissue and the first protrusion, a second protrusion extend from the internal surface of the housing a second distance resulting in a second contact pressure between the tissue and the second protrusion, a plurality of optical components comprise one or more light-transmitting components and one or more light-receiving components, the plurality of optical components comprising at least a first optical component disposed within the first protrusion, and at least a second optical component disposed within the second protrusion, one or more processors communicatively couple with the plurality of optical components, the one or more processors configured to, acquire physiological data from the user via at least a first optical channel including the first optical component, and a second optical channel including the second optical component, determine respective measurement quality metrics associated with the first optical channel and the second optical channel based at least in part on the physiological data, select the first optical channel or the second optical channel based at least in part on a comparison of the respective measurement quality metrics, and acquire additional physiological data using the first optical channel or the second optical channel based at least in part on the selecting.

Another apparatus is described. The apparatus may include means for a housing comprising an external surface and an internal surface, the internal surface configured to at least partially contact a tissue of a user, means for a first protrusion extending from the internal surface of the housing a first distance resulting in a first contact pressure between the tissue and the first protrusion, means for a second protrusion extending from the internal surface of the housing a second distance resulting in a second contact pressure between the tissue and the second protrusion, means for a plurality of optical components comprising one or more light-transmitting components and one or more light-receiving components, the plurality of optical components comprising at least a first optical component disposed within the first protrusion, and at least a second optical component disposed within the second protrusion, means for one or more processors communicatively coupled with the plurality of optical components, the one or more processors configured to, means for acquire physiological data from the user via at least a first optical channel including the first optical component, and a second optical channel including the second optical component, means for determine respective measurement quality metrics associated with the first optical channel and the second optical channel based at least in part on the physiological data, means for select the first optical channel or the second optical channel based at least in part on a comparison of the respective measurement quality metrics, and means for acquire additional physiological data using the first optical channel or the second optical channel based at least in part on the selecting.

A non-transitory computer-readable medium storing code is described. The code may include instructions executable by one or more processors to a housing comprise an external surface and an internal surface, the internal surface configured to at least partially contact a tissue of a user, a first protrusion extend from the internal surface of the housing a first distance resulting in a first contact pressure between the tissue and the first protrusion, a second protrusion extend from the internal surface of the housing a second distance resulting in a second contact pressure between the tissue and the second protrusion, a plurality of optical components comprise one or more light-transmitting components and one or more light-receiving components, the plurality of optical components comprising at least a first optical component disposed within the first protrusion, and at least a second optical component disposed within the second protrusion, one or more processors communicatively couple with the plurality of optical components, the one or more processors configured to, acquire physiological data from the user via at least a first optical channel including the first optical component, and a second optical channel including the second optical component, determine respective measurement quality metrics associated with the first optical channel and the second optical channel based at least in part on the physiological data, select the first optical channel or the second optical channel based at least in part on a comparison of the respective measurement quality metrics, and acquire additional physiological data using the first optical channel or the second optical channel based at least in part on the selecting.

In some examples of the method, apparatus, and non-transitory computer-readable medium described herein, and the method, apparatuses, and non-transitory computer-readable medium may include further operations, features, means, or instructions for wherein the first optical component comprises a first light-emitting component disposed within the first protrusion, wherein the first light-emitting component may be configured to emit first light associated with a first wavelength that penetrates the tissue of the user to a first penetration depth based at least in part on the first contact pressure and wherein the second optical component comprises a second light-emitting component disposed within the second protrusion, wherein the second light-emitting component may be configured to emit second light associated with the first wavelength that penetrates the tissue of the user to a second penetration depth based at least in part on the second contact pressure.

In some examples of the method, apparatus, and non-transitory computer-readable medium described herein, the first optical channel comprises the first light-emitting component and a light-receiving component, the second optical channel comprises the second light-emitting component and the light-receiving component, and a first distance between the first light-emitting component and the light-receiving component may be equal to a second distance between the second light-emitting component and the light-receiving component.

In some examples of the method, apparatus, and non-transitory computer-readable medium described herein, the wearable device comprises a wearable ring device, the first protrusion may be positioned at a first radial position along an inner curved surface of the wearable ring device, the second protrusion may be positioned at a second radial position along the inner curved surface of the wearable ring device, and the light-receiving component may be positioned at a third radial position along the inner curved surface of the wearable ring device that may be between the first and second radial positions.

In some examples of the method, apparatus, and non-transitory computer-readable medium described herein, the first light-emitting component, the second light-emitting component, or both, may be further configured to transmit light associated with a second wavelength.

Some examples of the method, apparatus, and non-transitory computer-readable medium described herein may further include operations, features, means, or instructions for transmit, using the first light-emitting component within the first protrusion, the first light associated with the first wavelength, transmit, using the second light-emitting component within the second protrusion, the second light associated with the first wavelength, receive the first light and the second light with the one or more light-receiving components of the plurality of optical components, wherein the one or more processors may be further configured to, determine a pressure gradient associated with the tissue of the user based at least in part on receiving the first light and the second light and based at least in part on the first contact pressure and the second contact pressure, and determine one or more physiological parameters associated with the user based at least in part on the pressure gradient.

In some examples of the method, apparatus, and non-transitory computer-readable medium described herein, the one or more physiological parameters comprise a blood pressure metric, a cardiovascular age metric, or both.

In some examples of the method, apparatus, and non-transitory computer-readable medium described herein, a third protrusion extending from the internal surface of the housing a third distance resulting in a third contact pressure between the tissue and the third protrusion, wherein the plurality of optical components further comprise at least a third optical component disposed within the third protrusion.

In some examples of the method, apparatus, and non-transitory computer-readable medium described herein, the one or more processors may be further configured to determine respective power consumption metrics associated with the first optical channel and the second optical channel based at least in part on acquiring the physiological data and the selecting may be based at least in part on the respective power consumption metrics.

In some examples of the method, apparatus, and non-transitory computer-readable medium described herein, the respective measurement quality metrics associated with the first optical channel and the second optical channel may be based at least in part on the first contact pressure and the second contact pressure.

In some examples of the method, apparatus, and non-transitory computer-readable medium described herein, and the method, apparatuses, and non-transitory computer-readable medium may include further operations, features, means, or instructions for determine a first signal quality difference between the first physiological data acquired using the first optical channel and the first physiological data acquired using the second optical channel, acquire second physiological data from the user during a second time interval subsequent to the first time interval, the second physiological data acquired via at least the first optical channel including the first optical component and the second optical channel including the second optical component, determine a second signal quality difference between the second physiological data acquired using the first optical channel and the second physiological data acquired using the second optical channel, and calibrate the second physiological data based at least in part on a comparison between the first signal quality difference and the second signal quality difference.

In some examples of the method, apparatus, and non-transitory computer-readable medium described herein, the one or more processors may be further configured to estimate one or more contact pressure changes at the first protrusion, the second protrusion, or both, between the first time interval and the second time interval based at least in part on the comparison between the first signal quality difference and the second signal quality difference and the calibrating may be based at least in part on the one or more contact pressure changes.

In some examples of the method, apparatus, and non-transitory computer-readable medium described herein, the first protrusion comprises a first light-blocking element extending a first height from the internal surface of the housing and the second protrusion comprises a second light-blocking element extending a second height from the internal surface of the housing.

In some examples of the method, apparatus, and non-transitory computer-readable medium described herein, the wearable device comprises a wearable ring device, the first protrusion may be positioned at a first radial position along an inner curved surface of the wearable ring device, and the second protrusion may be positioned at a second radial position along the inner curved surface of the wearable ring device.

In some examples of the method, apparatus, and non-transitory computer-readable medium described herein, the wearable device comprises a wrist-worn wearable device.

A method by an apparatus is described. The method may include a housing comprising an external surface and an internal surface, the internal surface configured to at least partially contact a tissue of a user, a first protrusion extending from the internal surface of the housing a first distance resulting in a first contact pressure between the tissue and the first protrusion, a second protrusion extending from the internal surface of the housing a second distance resulting in a second contact pressure between the tissue and the second protrusion, a first light-emitting component disposed within the first protrusion, wherein the first light-emitting component is configured to emit first light associated with a first wavelength that penetrates the tissue of the user to a first penetration depth based at least in part on the first contact pressure, a second light-emitting component disposed within the second protrusion, wherein the second light-emitting component is configured to emit second light associated with the first wavelength that penetrates the tissue of the user to a second penetration depth based at least in part on the second contact pressure, and one or more light-receiving components configured to receive light emitted by the first light-emitting component, the second light-emitting component, or both.

An apparatus is described. The apparatus may include one or more memories storing processor executable code, and one or more processors coupled with the one or more memories. The one or more processors may individually or collectively be operable to execute the code to cause the apparatus to a housing comprise an external surface and an internal surface, the internal surface configured to at least partially contact a tissue of a user, a first protrusion extend from the internal surface of the housing a first distance resulting in a first contact pressure between the tissue and the first protrusion, a second protrusion extend from the internal surface of the housing a second distance resulting in a second contact pressure between the tissue and the second protrusion, a first light-emit component disposed within the first protrusion, wherein the first light-emitting component is configured to emit first light associated with a first wavelength that penetrates the tissue of the user to a first penetration depth based at least in part on the first contact pressure, a second light-emit component disposed within the second protrusion, wherein the second light-emitting component is configured to emit second light associated with the first wavelength that penetrates the tissue of the user to a second penetration depth based at least in part on the second contact pressure, and one or more light-receive components configured to receive light emitted by the first light-emitting component, the second light-emitting component, or both.

Another apparatus is described. The apparatus may include means for a housing comprising an external surface and an internal surface, the internal surface configured to at least partially contact a tissue of a user, means for a first protrusion extending from the internal surface of the housing a first distance resulting in a first contact pressure between the tissue and the first protrusion, means for a second protrusion extending from the internal surface of the housing a second distance resulting in a second contact pressure between the tissue and the second protrusion, means for a first light-emitting component disposed within the first protrusion, wherein the first light-emitting component is configured to emit first light associated with a first wavelength that penetrates the tissue of the user to a first penetration depth based at least in part on the first contact pressure, means for a second light-emitting component disposed within the second protrusion, wherein the second light-emitting component is configured to emit second light associated with the first wavelength that penetrates the tissue of the user to a second penetration depth based at least in part on the second contact pressure, and means for one or more light-receiving components configured to receive light emitted by the first light-emitting component, the second light-emitting component, or both.

A non-transitory computer-readable medium storing code is described. The code may include instructions executable by one or more processors to a housing comprise an external surface and an internal surface, the internal surface configured to at least partially contact a tissue of a user, a first protrusion extend from the internal surface of the housing a first distance resulting in a first contact pressure between the tissue and the first protrusion, a second protrusion extend from the internal surface of the housing a second distance resulting in a second contact pressure between the tissue and the second protrusion, a first light-emit component disposed within the first protrusion, wherein the first light-emitting component is configured to emit first light associated with a first wavelength that penetrates the tissue of the user to a first penetration depth based at least in part on the first contact pressure, a second light-emit component disposed within the second protrusion, wherein the second light-emitting component is configured to emit second light associated with the first wavelength that penetrates the tissue of the user to a second penetration depth based at least in part on the second contact pressure, and one or more light-receive components configured to receive light emitted by the first light-emitting component, the second light-emitting component, or both.

In some examples of the method, apparatus, and non-transitory computer-readable medium described herein, the first light-emitting component, the second light-emitting component, or both, may be further configured to transmit light associated with a second wavelength.

In some examples of the method, apparatus, and non-transitory computer-readable medium described herein, the one or more light-receiving components comprise a first light-receiving component and a first distance between the first light-emitting component and the first light-receiving component may be equal to a second distance between the second light-emitting component and the first light-receiving component.

In some examples of the method, apparatus, and non-transitory computer-readable medium described herein, the wearable device comprises a wearable ring device, the first protrusion may be positioned at a first radial position along an inner curved surface of the wearable ring device, the second protrusion may be positioned at a second radial position along the inner curved surface of the wearable ring device, and the first light-receiving component may be positioned at a third radial position along the inner curved surface of the wearable ring device that may be between the first and second radial positions.

In some examples of the method, apparatus, and non-transitory computer-readable medium described herein, one or more processors communicatively coupled with the first light-emitting component, the second light-emitting component, and the one or more light-receiving components, wherein the one or more processors may be configured to, transmit, using the first light-emitting component within the first protrusion, the first light associated with the first wavelength, transmit, using the second light-emitting component within the second protrusion, the second light associated with the first wavelength, receive the first light and the second light with the one or more light-receiving components, determine a pressure gradient associated with the tissue of the user based at least in part on receiving the first light and the second light and based at least in part on the first contact pressure and the second contact pressure, and determine one or more physiological parameters associated with the user based at least in part on the pressure gradient.

In some examples of the method, apparatus, and non-transitory computer-readable medium described herein, the one or more physiological parameters comprise a blood pressure metric, a cardiovascular age metric, or both.

In some examples of the method, apparatus, and non-transitory computer-readable medium described herein, a third protrusion extending from the internal surface of the housing a third distance resulting in a third contact pressure between the tissue and the third protrusion and a third light-emitting component disposed within the third protrusion.

In some examples of the method, apparatus, and non-transitory computer-readable medium described herein, one or more processors communicatively coupled with the first light-emitting component, the second light-emitting component, and the one or more light-receiving components, wherein the one or more processors may be configured to, acquire first physiological data using the first light-emitting component and the one or more light-receiving components, acquire second physiological data using the second light-emitting component and the one or more light-receiving components, determine a first measurement quality metric and a second measurement quality metric associated with the first physiological data and the second physiological data, respectively, select the first light-emitting component or the second light-emitting component based at least in part on a comparison between the first measurement quality metric and the second measurement quality metric, and acquire additional physiological data using the first light-emitting component or the second light-emitting component based at least in part on the selecting.

In some examples of the method, apparatus, and non-transitory computer-readable medium described herein, the one or more processors may be further configured to determine a first power consumption metric and a second power consumption metric associated with the first physiological data and the second physiological data, respectively and the selecting may be based at least in part on a comparison between the first power consumption metric and the second power consumption metric.

In some examples of the method, apparatus, and non-transitory computer-readable medium described herein, the first measurement quality metric and the second measurement quality metric may be based at least in part on the first contact pressure and the second contact pressure, respectively.

In some examples of the method, apparatus, and non-transitory computer-readable medium described herein, one or more processors communicatively coupled with the first light-emitting component, the second light-emitting component, and the one or more light-receiving components, wherein the one or more processors may be configured to, acquire first physiological data from the user during a first time interval, the first physiological data acquired using light transmitted by the first light-emitting component and the second light-emitting component and received by the one or more light-receiving components, determine a first signal quality difference between the first physiological data acquired using the first light-emitting component and the first physiological data acquired using second light-emitting component, acquire second physiological data from the user during a second time interval subsequent to the first time interval, the second physiological data acquired using light transmitted by the first light-emitting component and the second light-emitting component and received by the one or more light-receiving components, determine a second signal quality difference between the second physiological data acquired using the first light-emitting component and the second physiological data acquired using the second light-emitting component, and calibrate the second physiological data based at least in part on a comparison between the first signal quality difference and the second signal quality difference.

In some examples of the method, apparatus, and non-transitory computer-readable medium described herein, the one or more processors may be further configured to estimate one or more contact pressure changes at the first protrusion, the second protrusion, or both, between the first time interval and the second time interval based at least in part on the comparison between the first signal quality difference and the second signal quality difference and the calibrating may be based at least in part on the one or more contact pressure changes.

In some examples of the method, apparatus, and non-transitory computer-readable medium described herein, the first protrusion comprises a first light-blocking element extending a first height from the internal surface of the housing and the second protrusion comprises a second light-blocking element extending a second height from the internal surface of the housing.

In some examples of the method, apparatus, and non-transitory computer-readable medium described herein, the wearable device comprises a wearable ring device, the first protrusion may be positioned at a first radial position along an inner curved surface of the wearable ring device, and the second protrusion may be positioned at a second radial position along the inner curved surface of the wearable ring device.

In some examples of the method, apparatus, and non-transitory computer-readable medium described herein, the wearable device comprises a wrist-worn wearable device.

The various illustrative blocks and modules described in connection with the disclosure herein may be implemented or performed with a general-purpose processor, a DSP, an ASIC, an FPGA or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration).

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope of the disclosure and appended claims. For example, due to the nature of software, functions described above can be implemented using software executed by a processor, hardware, firmware, hardwiring, or combinations of any of these. Features implementing functions may also be physically located at various positions, including being distributed such that portions of functions are implemented at different physical locations. Also, as used herein, including in the claims, “or” as used in a list of items (for example, a list of items prefaced by a phrase such as “at least one of” or “one or more of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same manner as the phrase “based at least in part on.”

Computer-readable media includes both non-transitory computer storage media and communication media including any medium that facilitates transfer of a computer program from one place to another. A non-transitory storage medium may be any available medium that can be accessed by a general purpose or special purpose computer. By way of example, and not limitation, non-transitory computer-readable media can comprise RAM, ROM, electrically erasable programmable ROM (EEPROM), compact disk (CD) ROM or other optical disk storage, magnetic disk storage or other magnetic storage devices, or any other non-transitory medium that can be used to carry or store desired program code means in the form of instructions or data structures and that can be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, include CD, laser disc, optical disc, digital versatile disc (DVD), floppy disk and Blu-ray disc where disks usually reproduce data magnetically, while discs reproduce data optically with lasers. Combinations of the above are also included within the scope of computer-readable media.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein, but is to be accorded the broadest scope consistent with the principles and novel features disclosed herein.

What is claimed is:

1. A wearable device, comprising:
a housing comprising an external surface and an internal surface, the internal surface configured to at least partially contact a tissue of a user;
a first protrusion extending from the internal surface of the housing a first distance resulting in a first contact pressure between the tissue and the first protrusion;
a second protrusion extending from the internal surface of the housing a second distance resulting in a second contact pressure between the tissue and the second protrusion;
a plurality of optical components comprising one or more light-transmitting components and one or more light-receiving components, the plurality of optical components comprising at least a first optical component disposed within the first protrusion, and at least a second optical component disposed within the second protrusion; and
one or more processors communicatively coupled with the plurality of optical components, the one or more processors configured to:
acquire physiological data from the user via at least a first optical channel including the first optical component, and a second optical channel including the second optical component;
determine respective measurement quality metrics associated with the first optical channel and the second optical channel based at least in part on the physiological data;
select the first optical channel or the second optical channel based at least in part on a comparison of the respective measurement quality metrics; and
acquire additional physiological data using the first optical channel or the second optical channel based at least in part on the selecting.

2. The wearable device of claim 1,
wherein the first optical component comprises a first light-emitting component disposed within the first protrusion, wherein the first light-emitting component is configured to emit first light associated with a first wavelength that penetrates the tissue of the user to a first penetration depth based at least in part on the first contact pressure, and
wherein the second optical component comprises a second light-emitting component disposed within the second protrusion, wherein the second light-emitting component is configured to emit second light associated with the first wavelength that penetrates the tissue of the user to a second penetration depth based at least in part on the second contact pressure.

3. The wearable device of claim 2, wherein the first optical channel comprises the first light-emitting component and a light-receiving component, and wherein the second optical channel comprises the second light-emitting component and the light-receiving component, wherein a first distance between the first light-emitting component and the light-receiving component is equal to a second distance between the second light-emitting component and the light-receiving component.

4. The wearable device of claim 3, wherein the wearable device comprises a wearable ring device, wherein the first protrusion is positioned at a first radial position along an inner curved surface of the wearable ring device, and wherein the second protrusion is positioned at a second radial position along the inner curved surface of the wearable ring device, and wherein the light-receiving component is positioned at a third radial position along the inner curved surface of the wearable ring device that is between the first and second radial positions.

5. The wearable device of claim 2, wherein the first light-emitting component, the second light-emitting component, or both, are further configured to transmit light associated with a second wavelength.

6. The wearable device of claim 2, wherein, to acquire the physiological data, the one or more processors are configured to:
transmit, using the first light-emitting component within the first protrusion, the first light associated with the first wavelength;
transmit, using the second light-emitting component within the second protrusion, the second light associated with the first wavelength; and
receive the first light and the second light with the one or more light-receiving components of the plurality of optical components, wherein the one or more processors are further configured to:
determine a pressure gradient associated with the tissue of the user based at least in part on receiving the first light and the second light and based at least in part on the first contact pressure and the second contact pressure; and
determine one or more physiological parameters associated with the user based at least in part on the pressure gradient.

7. The wearable device of claim 6, wherein the one or more physiological parameters comprise a blood pressure metric, a cardiovascular age metric, or both.

8. The wearable device of claim 1, further comprising:
a third protrusion extending from the internal surface of the housing a third distance resulting in a third contact pressure between the tissue and the third protrusion, wherein the plurality of optical components further comprise at least a third optical component disposed within the third protrusion.

9. The wearable device of claim 1, wherein the one or more processors are further configured to:
determine respective power consumption metrics associated with the first optical channel and the second optical channel based at least in part on acquiring the physiological data, wherein the selecting is based at least in part on the respective power consumption metrics.

10. The wearable device of claim 1, wherein the respective measurement quality metrics associated with the first optical channel and the second optical channel are based at least in part on the first contact pressure and the second contact pressure.

11. The wearable device of claim 1, wherein the physiological data comprises first physiological data acquired during a first time interval, wherein the one or more processors are further configured to:
determine a first signal quality difference between the first physiological data acquired using the first optical channel and the first physiological data acquired using the second optical channel;
acquire second physiological data from the user during a second time interval subsequent to the first time interval, the second physiological data acquired via at least the first optical channel including the first optical component and the second optical channel including the second optical component;
determine a second signal quality difference between the second physiological data acquired using the first optical channel and the second physiological data acquired using the second optical channel; and
calibrate the second physiological data based at least in part on a comparison between the first signal quality difference and the second signal quality difference.

12. The wearable device of claim 11, wherein the one or more processors are further configured to estimate one or more contact pressure changes at the first protrusion, the second protrusion, or both, between the first time interval and the second time interval based at least in part on the comparison between the first signal quality difference and the second signal quality difference, wherein the calibrating is based at least in part on the one or more contact pressure changes.

13. The wearable device of claim 1, wherein the first protrusion comprises a first light-blocking element extending a first height from the internal surface of the housing, and wherein the second protrusion comprises a second light-blocking element extending a second height from the internal surface of the housing.

14. The wearable device of claim 1, wherein the wearable device comprises a wrist-worn wearable device.

15. A method, comprising:
acquiring first physiological data from a user via a first optical channel of a wearable device, the first optical channel comprising at least a first optical component disposed at least partially within a first protrusion extending from a surface of the wearable device a first distance resulting in a first contact pressure between a tissue of the user and the first protrusion;
acquiring second physiological data from the user via a second optical channel of a wearable device, the second optical channel comprising at least a second optical component disposed at least partially within a second protrusion extending from the surface of the wearable device a second distance resulting in a second contact pressure between the tissue of the user and the second protrusion;
determining respective measurement quality metrics associated with the first optical channel and the second optical channel based at least in part on the first physiological data and the second physiological data;
selecting the first optical channel or the second optical channel based at least in part on a comparison of the respective measurement quality metrics; and
acquiring additional physiological data using the first optical channel or the second optical channel based at least in part on the selecting.

16. The method of claim 15,
wherein the first optical component comprises a first light-emitting component disposed within the first protrusion, wherein the first light-emitting component is configured to emit first light associated with a first wavelength that penetrates the tissue of the user to a first penetration depth based at least in part on the first contact pressure, and
wherein the second optical component comprises a second light-emitting component disposed within the second protrusion, wherein the second light-emitting component is configured to emit second light associated with the first wavelength that penetrates the tissue of the user to a second penetration depth based at least in part on the second contact pressure.

17. The method of claim 15, further comprising:
determining a pressure gradient associated with the tissue of the user based at least in part on acquiring the first physiological data and the second physiological data based at least in part on the first contact pressure and the second contact pressure; and
determining one or more physiological parameters associated with the user based at least in part on the pressure gradient.

18. The method of claim 17, wherein the one or more physiological parameters comprise a blood pressure metric, a cardiovascular age metric, or both.

19. A wearable device, comprising:
a housing comprising an external surface and an internal surface, the internal surface configured to at least partially contact a tissue of a user;
a first protrusion extending from the internal surface of the housing a first distance resulting in a first contact pressure between the tissue and the first protrusion;
a second protrusion extending from the internal surface of the housing a second distance resulting in a second contact pressure between the tissue and the second protrusion;
a first light-emitting component disposed within the first protrusion, wherein the first light-emitting component is configured to emit first light associated with a first wavelength that penetrates the tissue of the user to a first penetration depth based at least in part on the first contact pressure;
a second light-emitting component disposed within the second protrusion, wherein the second light-emitting component is configured to emit second light associated with the first wavelength that penetrates the tissue of the user to a second penetration depth based at least in part on the second contact pressure; and
one or more light-receiving components configured to receive light emitted by the first light-emitting component, the second light-emitting component, or both.

20. The wearable device of claim 19, wherein the first light-emitting component, the second light-emitting component, or both, are further configured to transmit light associated with a second wavelength.
